(19) United States
(12) Patent Application Publication
(10) Pub. No.: US 2024/0389955 A1
(43) Pub. Date: Nov. 28, 2024

(54) HYBRID METHOD FOR FAST FUNCTIONAL IMAGING WITH SPARSE SAMPLING IN TOMOGRAPHY

(71) Applicant: California Institute of Technology, Pasadena, CA (US)

(72) Inventors: Peng Hu, Alhambra, CA (US); Lihong Wang, Arcadia, CA (US)

(21) Appl. No.: 18/658,823

(22) Filed: May 8, 2024

Related U.S. Application Data
(60) Provisional application No. 63/464,812, filed on May 8, 2023.
(60) Provisional application No. 63/464,832, filed on May 8, 2023.

(51) Int. Cl. A61B 6/03; A61B 6/00; A61B 6/58

(52) U.S. Cl. CPC A61B 6/032; A61B 6/5247; A61B 6/58

(57) ABSTRACT

Certain aspects pertain to methods and systems for compressing tomographic imaging system matrices and/or functional tomographic imaging.

### CROSS-REFERENCES TO RELATED APPLICATIONS

This application claims priority to and benefit of U.S. Provisional Patent Application No. 63/464,812, titled “A Hybrid Method For Fast Functional Imaging With Sparse Sampling In Tomography,” filed on May 8, 2023 and U.S. Provisional Patent Application No. 63/464,832, titled “Intra-Image Nonrigid Motion Correction In Tomography,” filed on May 8, 2023; both of which are hereby incorporated by reference in their entireties and for all purposes.

### FEDERALLY SPONSORED RESEARCH OR DEVELOPMENT

This invention was made with government support under Grant No(s). NS102213 & EB029823 & CA220436 awarded by the National Institutes of Health. The government has certain rights in the invention.

### FIELD

Certain aspects generally tomography, and more specifically to, methods for compression of system matrices and/or functional imaging in tomography.

### BACKGROUND

Tomographic imaging modalities x-ray computed tomography (CT), magnetic resonance imaging (MRI), and photoacoustic computed tomography (PACT) produce cross-sectional images of tissue by detection of penetrating x-rays, nuclear-magnetic-resonance-induced radio waves, and light absorption-induced ultrasonic waves, respectively. Each modality with a certain setup is described by a system matrix. Tomographic imaging modalities are generally described by large system matrices. Sparse sampling can degrade the system matrix and image quality. Accurate image reconstruction poses requirements to the system matrix, which are often violated. For example, to achieve high temporal resolution for functional imaging in tomography, the spatial sampling density is often sacrificed, which introduces artifacts in the reconstructed image and may affect the functional signal extraction.

Various methods have been proposed to compensate for system-matrix imperfections from image-domain, signal-domain, and cross-domain perspectives. However, due to the large size of each system matrix, these methods tend not to manipulate or correct the system matrix directly and have limitations. For example, for sparse sampling functional imaging, traditional methods can mitigate artifacts in images but their performance drops sharply as sampling density reduces. Deep neural networks (DNNs) show high performance in mitigating artifacts but tend to generate false image features when the sampling density is low, and DNNs require imaging-modality- and device-dependent datasets, which are not always available. Moreover, most of these existing methods introduce nonlinearity while mitigating artifacts, which disrupts the functional signals that are often much weaker than background signals.

### SUMMARY

Techniques disclosed herein may be practiced with a processor-implemented method, a system comprising one or more processors and one or more processor-readable media, and/or one or more non-transitory processor-readable media.

Certain embodiments pertain to tomographic imaging system matrix compression methods. In some cases, a method includes acquiring a plurality of detected tomographic system responses and shifting one or more of the detected tomographic system responses to align arrival times. For each virtual sensor of a plurality of virtual sensors, the method determines an approximate system response that is a linear combination of a plurality of basis functions. The approximate system response is determined by performing a singular value decomposition (SVD) process on the aligned, detected tomographic system responses to obtain a plurality of coefficients for the linear combination. The method determines a compressed system matrix including approximate system responses for all virtual sensors from the plurality of coefficients determined for each of the virtual sensors.

Certain embodiments pertain to systems that include a tomographic imaging device comprising one or more sensor arrays and one or more processors in electrical communication with the tomographic imaging device. The one or more processors one or more processor-readable media store instructions which, when executed by the one or more processors, cause performance of (a) performing piece-wise compression of a plurality of aligned system responses to generate a compressed system matrix, (b) applying the compressed system matrix to generate a plurality of simulated virtual sensor signals; and (c) performing an iterative reconstruction procedure to reconstruct tomographic image from detected sensor signals by comparing a difference between the simulated virtual sensor signals and the detected sensor signals.

Certain embodiments pertain to methods for functional tomographic imaging. In some cases, a method performs iterative reconstruction of a smooth modulation image using a set of sparsely sampled functional signals from a plurality of sensors of a tomographic imaging device. The method obtains a modulated prior image by upsampling the smooth modulation image and multiplying the upsampled smooth modulation image by a densely sampled prior image. The method applies a dense-sampling system matrix to the modulated prior image to determine a dense forward solution and reconstructing a modulated UBP image from the dense forward solution using UBP. The method applies a sparse sampling system matrix to the modulated prior image to determine a sparsely sampled solution and reconstructing a residual image using UBP from a difference between the set of sparsely sampled functional signals and the sparsely sampled solution. The method determines a hybrid functional image by adding the modulated UBP image to the residual image.

Certain embodiments pertain to systems including a tomographic imaging device comprising one or more sensor arrays and one or more processors in electrical communication with the tomographic imaging device. The one or more processor-readable media store instructions which, when executed by the one or more processors, cause performance of (a) performing iterative reconstruction of a smooth modulation image using a set of sparsely sampled functional signals from a plurality of sensors of a tomographic imaging device, (b) obtaining a modulated prior image by upsampling the smooth modulation image and multiplying the upsampled smooth modulation image by a densely sampled prior image, (c) applying a dense-sampling system matrix to the modulated prior image to determine a dense forward solution from the dense forward solution signal and reconstructing a modulated UBP image from the dense forward solution using UBP, and (d) applying a sparse sampling system matrix to the modulated prior image to determine a sparsely sampled solution and reconstructing a residual image from a difference between the set of sparsely sampled functional signals and the sparsely sampled solution; and (c) determining a hybrid functional image by adding the modulated UBP image to the residual image.

These and other features are described in more detail below with reference to the associated drawings.

### BRIEF DESCRIPTION OF THE DRAWINGS

FIG. 1A depicts an illustration of a virtual transducer hemispherical array formed by rotation of arc arrays of a PACT system and exemplary responses of three virtual sensor elements to signals from three voxels, according to an embodiment.

FIG. 1B depicts an illustration of piece-wise compression of a tomographic imaging system matrix compression (TISMC) method, according to an embodiment.

FIG. 2A is an illustration depicting the forward simulation procedure of a TISMC method that simulates sensor signals, according to embodiments.

FIG. 2B depicts an illustration of system matrix slicing in voxel indices and virtual sensor element indices, according to an embodiment.

FIG. 3 depicts a flowchart illustrating operations of a TISMC method, according to various embodiments.

FIG. 4 depicts a flowchart illustrating operations of a hybrid method, according to various embodiments.

FIG. 5 depicts images reconstructed by UBP, regularized iterative method, and hybrid method from signals detected at sparsely distributed elements, according to an embodiment.

FIG. 6A depicts structural similarity index measures (SSIMs) between the reconstructed images in FIG. 5 for the three methods: UBP, regularized iterative method, and hybrid method, according to an embodiment.

FIG. 6B depicts illustrations of results of linearity tests of the three methods: UBP, regularized iterative method, and hybrid method, according to an embodiment.

FIG. 7 depicts a densely sampled image of a mouse brain reconstructed by UBP and sparsely sampled images of the mouse brain reconstructed using UBP, a regularized iterative method, and an example of a hybrid method, according to an embodiment.

FIG. 8A depicts an illustration of an electrical stimulation procedure applied to a mouse's right front paw at five cycles, according to an implementation.

FIG. 8B depicts functional images obtained from the images reconstructed using, a regularized iterative method, and an example of a hybrid method according to an embodiment.

FIG. 9A depicts an illustration of a transducer element, a single point source, and a transducer element local coordinate system with axes \hat{x}ₙ, ŷₙ, and \hat{z}ₙ, according to an embodiment.

FIG. 9B depicts an illustration of four point sources in the local coordinate system of the transducer element in FIG. 9A, according to an embodiment.

FIG. 10A depicts a graph of four responses of the transducer element in FIG. 9B to the signals from the four point sources, according to an embodiment.

FIG. 10B depicts an illustration of four responses to the signals from four point sources in FIG. 9B using linear combinations of three temporal singular functions based on a singular value decomposition (SVD) procedure, according to an embodiment.

FIG. 11A depicts illustrations of responses of transducer element to fifty (50) point sources with decreasing distances, the temporally-shifted form, white noise responses, according to an embodiment.

FIG. 11B depicts a graph with normalized singular values in the SVDs of the signals, according to an embodiment.

FIG. 11C depicts a graph with proportions of the variances in the SVDs of the signals, according to an embodiment.

FIG. 12A depicts an illustration of a numerical phantom formed by three rectangular cuboids intersecting at their centers, according to an embodiment.

FIG. 12B depicts a virtual 2D array, four subdomains D₁, D₂, D₃, and D₄, and the domain occupied by rotations of the four subdomains around the array axis, according to an implementation.

FIG. 12C depicts a plot of computation times of the fast and slow operators for the forward simulations, according to an implementation.

FIG. 13A depicts images of relative errors of the simulated signals for all virtual elements with the numerical phantom in four subdomains D₁, D₂, D₃, and D₄, according to an implementation.

FIG. 13B depicts a comparison of the signals simulated by the fast and slow operators, according to an implementation.

FIG. 14 are plots of the relative error of a reconstructed image in subdomains D₁, D₂, D₃, and D₄ with 1 to 256 iterations, values on three lines in the reconstructed images with 1, 4, 8, 64, and 256 iterations, and a comparison between ground truth and the 256-iteration reconstructed values along the three lines, according to an implementation.

FIG. 15 includes plots of results of an example of iterative image reconstruction with noisy data, according to an embodiment.

FIG. 16 depicts an illustration including regularized iterative reconstruction of mouse brain images with sparse sampling, according to an implementation.

FIG. 17 depicts a flowchart of a hybrid method for image reconstruction from sparsely sampled raw data and a prior image, according to an embodiment.

FIG. 18 depicts maximum amplitude projections (MAPs) of the images shown in FIG. 5.

FIG. 19A depicts images of first and second numerical phantoms and their sum, and an image domain and two virtual transducer arrays, according to an embodiment.

FIG. 19B depicts MAPs of images reconstructed using UBP from signals detected by the two virtual arrays in FIG. 19A, and MAPs of the linear test residuals, according to an embodiment.

FIG. 19C depicts MAPs of images reconstructed using a regularized iterative method from signals detected by the two virtual arrays in FIG. 19A, and the MAPs of the linear test residuals, according to an embodiment.

FIG. 20A depicts a ground-truth image used as a first prior image and a second prior image obtained by adding scattered point sources to the first one, according to an embodiment.

FIG. 20B depicts MAPs of images reconstructed using UBP from signals detected by two virtual transducer arrays, and the MAPs of the linear test residuals, according to an embodiment.

FIG. 20C depicts MAPs of the images reconstructed using an example of a hybrid method with the first and second prior images, respectively, from signals detected by the two virtual arrays, according to an embodiment.

FIG. 21A depicts an image of a background numerical phantom, an image of a functional numerical phantom, and a virtual array for functional imaging simulation, according to an embodiment.

FIG. 21B depicts a plot of modulation factors of the background phantom in FIG. 21A, according to an embodiment.

FIG. 21C depicts a plot of modulation factors of the functional phantom in FIG. 21A, according to an embodiment.

FIG. 22 depicts functional images extracted using a regularized-correlation-based method from ground-truth images and images reconstructed with UBP, the regularized iterative method, and an example of a hybrid method, according to an embodiment.

FIG. 23 depicts mouse brain functional images in vivo extracted using the regularized-correlation-based method from images reconstructed with UBP and an example of a hybrid method, according to an embodiment.

FIG. 24 depicts mouse brain functional images in vivo extracted using a regularized-correlation-based method from images reconstructed through a regularized iterative method according to an embodiment.

FIG. 25A depicts a block diagram of a tomographic imaging system having a computing system, according to various embodiments.

FIG. 25B depicts a block diagram of an example of a computing system, according to an embodiment.

These and other features are described in more detail below with reference to the associated drawings.

### DETAILED DESCRIPTION

Different aspects are described below with reference to the accompanying drawings. The features illustrated in the drawings may not be to scale. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments.

### I. Introduction

Certain embodiments pertain to methods for compressing tomographic imaging system matrices (also referred to herein as “tomographic imaging system matrix compression (TISMC) methods” or “compression methods”). In some cases, TISMC methods temporally shift one or more of the responses to align arrival times and then group aligned responses for each virtual sensor element. The TISMC methods then perform a grouped SVD procedure (for each virtual sensor element) on the aligned responses to approximate the responses as linear combinations of a plurality of basis functions to obtain a compressed system matrix (piece-wise compression). For each combination of voxel and virtual sensor element, the coefficients for the basis functions are applied with the original arrival times for each virtual sensor element and convolutions are performed (e.g., implemented by FFT) to obtain simulated sensor signals for all virtual sensor elements. An iterative reconstruction procedure is applied to obtain a tomographic image from the detected signals. These TISMC methods improve computational efficiency (e.g., 42 times), which can enable efficient system matrix slicing and manipulation. This efficiency allows us to perform a larger number (e.g., 10000) of data-driven manipulations of subsets of the system matrix to improve the image quality, which is too time-consuming using traditional methods.

Certain embodiments pertain to hybrid methods for fast functional imaging with sparse sampling in tomography (also sometimes referred to herein as “fast functional imaging methods” or “hybrid methods”). These hybrid methods incorporate a densely sampled prior image into the system matrix to reduce unknown variables during image reconstruction. These hybrid methods can perform fast functional imaging by reconstructing images from sparsely sampled signals using the system matrix that incorporates the densely sampled prior image. Incorporating the densely sampled prior image into the system matrix maintains linearity during image reconstruction while substantially mitigating artifacts, which can be critical for functional signal (typically much weaker than structure signals in a single image) extraction.

For sparse sampling functional imaging, traditional methods mitigate artifacts in images but their performances drop sharply as the sampling density reduces. Deep neural networks show high performance in mitigating artifacts but tend to generate false image features when the sampling density is low, and they require imaging-modality- and device-dependent datasets, which are not always available. Moreover, most of the methods introduce nonlinearity while mitigating artifacts, which disrupts the functional signals that are often much weaker than background signals.

In both numerical simulations and mouse brain functional imaging in vivo discussed herein, certain examples of hybrid methods have been shown to substantially mitigate artifacts in the reconstructed images and reduce false positive regions in the functional image, and its linearity is important for maintaining the true functional region. Due to its high robustness, hybrid methods can be used to accelerate or enhance the performance of an existing tomographic imaging system and reduce the cost of a future system for functional imaging.

Certain embodiments pertain to methods that combine a TISMC method and a hybrid method. For example, a method may employ a TISMC method to compress a tomographic system matrix. Enabled by this efficiency, the method may then perform operations of the hybrid method to incorporate a densely sampled prior image into the tomographic system matrix and construct a plurality of functional images from sparsely sampled signals.

### II. Methods

TISMC methods and hybrid methods described herein can be applied to various tomographic imaging techniques such as, e.g., a 3D photoacoustic computed tomography (3D PACT), x-ray computed tomography (CT) and magnetic resonance imaging (MRI). Section IV discussed the application of TISMC methods and hybrid methods to various tomographic imaging techniques in more detail. An example of a 3D PACT system is described in Lin, L. et al. “High-speed three-dimensional photoacoustic computed tomography for preclinical research and clinical translation,” Nat. Commun. Vol. 12, pp. 1-10 (2021), which is hereby incorporated by reference in its entirety and for all purposes. Another example of a 3D PACT system is described in U.S. patent application Ser. No. 16/798,204, titled “PHOTOACOUSTIC COMPUTED TOMOGRAPHY (PACT) SYSTEMS AND METHODS,” filed on Feb. 20, 2020, which is hereby incorporated by reference in its entirety and for all purposes.

In certain examples discussed herein, the 3D PACT system disclosed in Lin, L. et al. “High-speed three-dimensional photoacoustic computed tomography for preclinical research and clinical translation,” Nat. Commun. Vol. 12, pp. 1-10 (2021) is used to demonstrate examples of TISMC methods and/or hybrid methods. In many of these examples, the PACT system includes a four 256-element 2-D arc ultrasonic transducer arrays where each 2-D arc array has a central frequency of 2.25 MHz and one-way bandwidth of 98%. The 3D PACT system is used to demonstrate hybrid methods due to its representatively large size in tomography: light-absorption-induced ultrasonic wave from every voxel in an image is detected by every transducer element and the system matrix is intrinsically a 6D tensor. In certain examples, hybrid methods are applied to both numerical simulations and in vivo experiments: mouse brain functional imaging with sparse sampling demonstrating that these methods may substantially improve functional and structural image qualities.

### A. Tomographic Imaging System Matrix Compression (TISMC) Methods

According to various embodiments, a TISMC method can compress a tomographic imaging system response matrix, which may advantageously enable efficient system matrix slicing and manipulation. The TISMC method temporally shifts one or more of the system responses to align arrival times and then groups the aligned responses for each virtual sensor element. The TISMC method then performs a grouped SVD procedure (for each virtual sensor element) to the aligned responses to approximate the responses as linear combinations of a plurality of basis functions (e.g., three temporal singular functions) to obtain coefficients for a compressed system matrix (piece-wise compression). In some cases, the TISMC method may also include forward simulations. For example, for each combination of voxel and virtual sensor element, the coefficients for the basis functions may be applied with the original arrival times for each virtual sensor element and convolutions (e.g., three convolutions) are performed (e.g., implemented by FFT) to obtain simulated sensor signals for all virtual sensor elements. This fast forward operator can be used in an iterative reconstruction procedure to obtain a tomographic image from the detected signals. TISMC methods improve computational efficiency (e.g., up to 42 times), which can enable efficient system matrix slicing and manipulation.

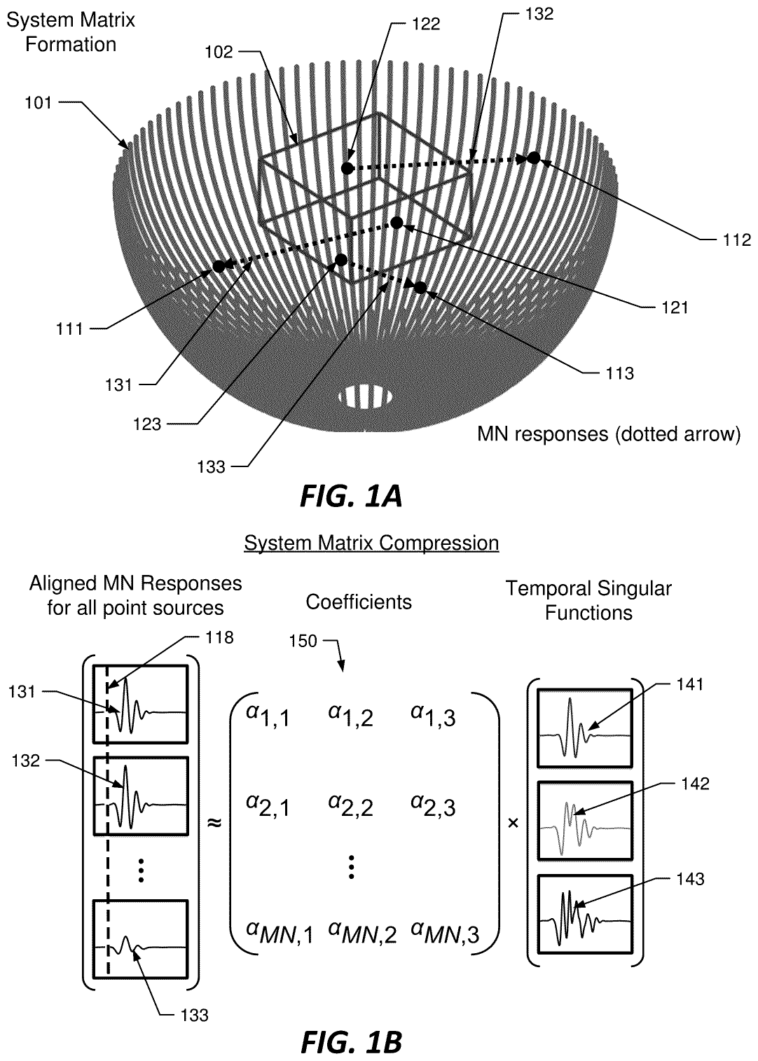

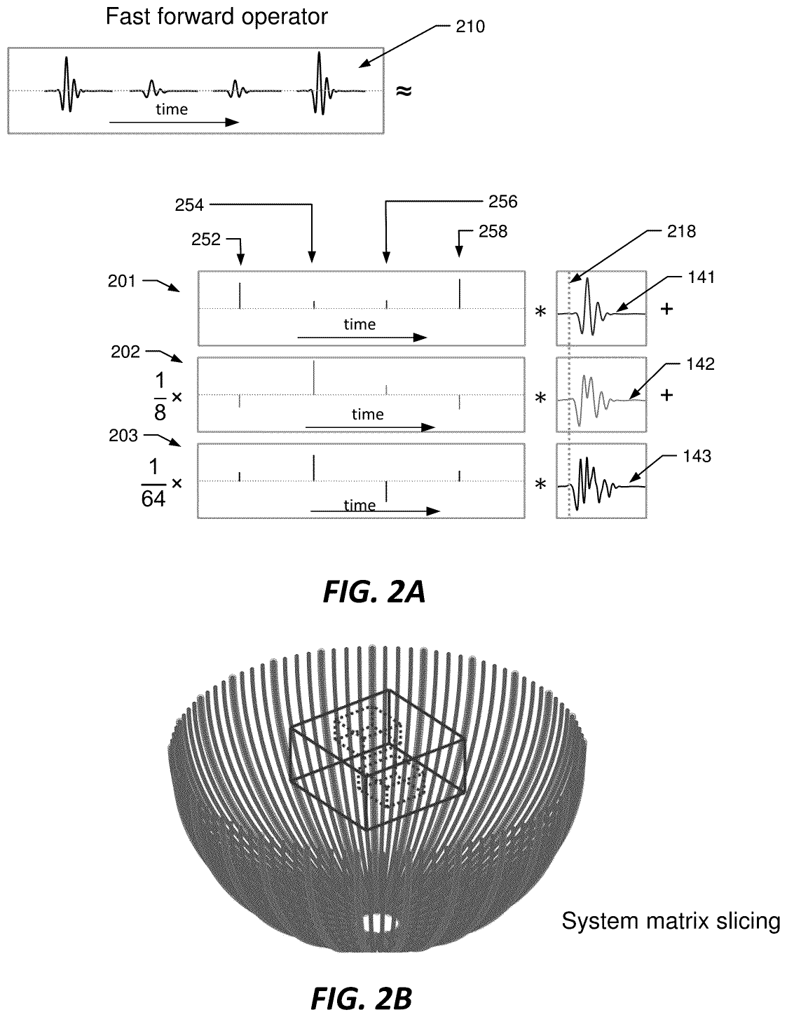

FIGS. 1A-1B and 2A-2B discuss an example of a TISMC method, according to an embodiment, that is used to compress a tomographic system matrix of the PACT system described in Lin, L. et al. “Highspeed three-dimensional photoacoustic computed tomography for preclinical research and clinical translation,” Nat. Commun. Vol. 12, pp. 1-10 (2021). The PACT system includes four 256-element arc ultrasonic transducer arrays where each arc array has a central frequency of 2.25 MHz and one-way bandwidth of 98%. The object being imaged was assumed to be a homogenous medium for simplicity. Although discussed with respect to a PACT system, it would be understood that the TISMC method also applies to other tomographic imaging systems such as CT and MRI systems.

FIG. 1A depicts an illustration of a virtual hemispherical array 101 formed by the rotation of the four 256-element arc transducer arrays in the PACT system. The illustration also shows an image domain with a 3D tomographic image 102 depicted as a rectangular cuboid with solid edges. The illustration also includes the paths of three system responses 131, 132, and 133 denoted by dotted arrows from three voxels 121, 122, and 123 (point sources) to three virtual transducer elements 111, 112, and 113. The virtual hemispherical array 101 is truncated at the bottom for light delivery. The detected ultrasonic signals from the four arc transducer arrays were used to form the 3D tomographic image 102.

The number of voxels in the 3-D image and the number of virtual sensor elements are denoted as M and N, respectively in various examples. During data acquisition, the physical sensors are rotated/translated while detecting signals at different locations. Virtual sensor elements are defined at the various detection locations of the physical sensors. A tomographic imaging system matrix is formed of MN responses of all N virtual sensor elements to the signals from all M voxels in the 3D tomographic image. For instance, in the example illustrated in FIG. 1A, the number of voxels in the 3D tomographic image 102 and the number of virtual transducer elements in the virtual hemispherical array are denoted as M and N, respectively. For illustration purposes, the paths of three responses 131, 132, and 133 are denoted by dotted arrows from three voxels (denoted as dots) 121, 122, and 123 to three virtual transducer elements (denoted as dots) 111, 112, and 113. Without compression techniques, the system matrix may be as large as, e.g., 600*600*300*400*256*151 non-zero elements It can be expensive to store and manipulate a system matrix with a large number of responses directly from memory and computation perspectives, respectively.

According to various embodiments, a TISMC method compresses the system matrix, which can advantageously enable efficient system matrix slicing and manipulation. First, considering that arrival times of the responses are determined by the voxel-element distances (i.e. distances between the voxel and the virtual transducer), TISMC methods of various embodiments may temporally shift one or more of the responses to align the arrival times to remove the differences in response arrival times. FIG. 1B depicts the three responses 131, 132, and 133 from FIG. 1A that are temporally shifted to align arrival times and the aligned responses grouped by sensor element are then compressed based on an SVD procedure, according to an embodiment. As shown, the first response 131, second response 132, . . . , MN response 133 have been temporally shifted to align along a dashed line 118 to remove the differences in arrival times from the voxels to elements, and the responses are compressed based on SVD with the first three temporal singular functions 141, 142, and 143, respectively. In other implementations, other basis functions may be used.

In various embodiments, a TISMC method temporally shifts one or more of the responses so that the arrival times are aligned to remove the differences in response arrival times and then groups the responses for each sensor element. The TISMC method then performs a grouped SVD procedure to the shifted responses to approximate the responses as linear combinations of a plurality of basis functions to obtain a compressed system matrix (piece-wise compression). The grouped SVD procedure obtains for each combination of source point and sensor element, a coefficient for each basis function that is used in the linear combinations. An example of basis functions that can be used are three temporal singular functions (e.g., three temporal singular functions 141, 142, and 143 shown in FIG. 1B).

For example, the TISMC method being implemented in FIG. 1B groups the responses for each virtual transducer element. The TISMC method then performs a grouped SVD procedure to the shifted responses to approximate the responses as linear combinations (where coefficients are shown in FIG. 1B) of the three temporal singular functions 141, 142, and 143 for efficient compression of the system matrix. Section III (A) has additional detail of the process of performing SVD to the responses grouped for each virtual element in the element's local coordinate system. Due to the homogeneous-medium assumption and identity of all elements in the system, SVDs for all virtual elements share the same set of temporal singular functions including the first three temporal singular functions 141, 142, and 143 shown in FIG. 1B. In other implementations, another plurality of basis functions may be used.

The system matrix compression of a TISMC method not only saves memory in storing the system matrix but also may enable fast and accurate forward simulation. Once the system matrix is compressed, the TISMC method can simulate the sensors signals using the plurality of coefficients obtained during piece-wise compression. The TISMC method determines a simulated signal at each virtual sensor element. For all the voxels, the TISMC method determines a time series of coefficients for each basis function based on the actual arrival times. After obtaining all the coefficients with different arrival times for each basis function for each virtual sensor element, the TISMC method performs a plurality of convolutions (e.g., implemented by FFT) on the time series of coefficients for the plurality of basis functions to obtain a simulated sensor signal for each virtual sensor. Based on the fast forward operator, an iterative reconstruction procedure can then be used to reconstruct one or more 3D images from the detected signals. An example of an iterative reconstruction procedure is the iterative gradient descent algorithm. Another example of a suitable iterative reconstruction procedure is the fast iterative shrinkage-thresholding algorithm FISTA, which can be found in Beck, A. & Teboulle, M. A, “fast iterative shrinkage-thresholding algorithm for linear inverse problems,” SIAM J. Imaging Sci. 2, 183-202 (2009), which is hereby incorporated by reference in its entirety. An example of a suitable iterative reconstruction procedure is the nonnegativity-constrained iterative method described by Eqn. 33.

FIG. 2A is an illustration depicting the forward simulation procedure of a TISMC method that simulates sensor signals, according to embodiments. In this example, for each combination of voxel and virtual sensor element, instead of adding the whole weighted response to the signals, only three coefficients are applied: values of the first three spatial singular functions 141, 142, and 143 from FIG. 1B.

For each of the voxels (point sources), the TISMC method determines a time series of coefficients for each basis function based on the original (unshifted) arrival times of the original system matrix. In the illustrated example, three temporal singular functions 141, 142, and 143 are being used and three time series of coefficients 201, 202, and 203 are obtained. The time series of coefficients 201, 202, and 203 include coefficients for a first point source (voxel) 252, coefficients for a second point source 254, coefficients for a third point source 256, and coefficients for a fourth point source 258. After obtaining all the coefficients with different arrival times for each virtual element, the TISMC method performs three convolutions (implemented by FFT) to the times series of coefficients to approximate the simulated signals. A compressed system matrix with the simulated signals using the original system matrix can then be formed. FIG. 2A includes a simulated signal 210 of a transducer element approximated by three convolutions with the three temporal singular functions 141, 142, and 143 where (zero) arrival time is indicated by a dotted line 218.

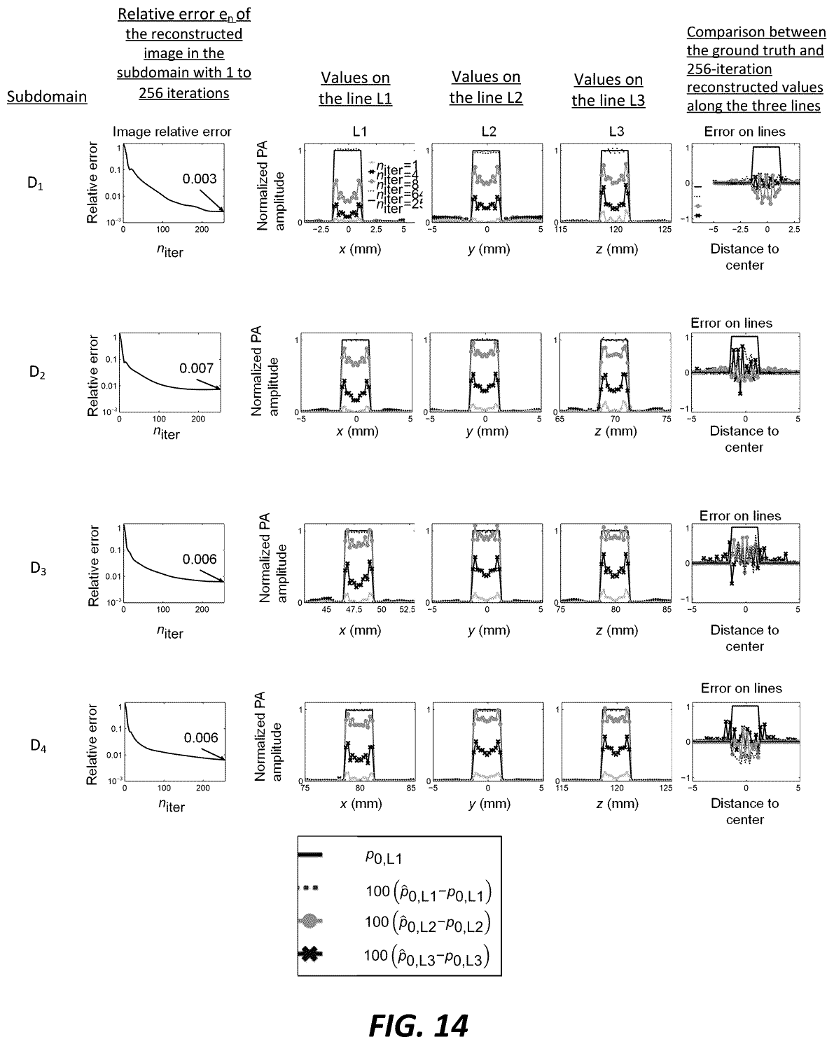

Based on the fast forward operator, one or more tomographic images can be reconstructed from the detected signals using an iterative reconstruction procedure. Images reconstructed iteratively using the fast forward operator from signals simulated by a traditional method (a slow operator) were observed to have negligible image-domain relative errors (maximum value of 0.007) as shown in FIG. 14 and discussed in Section III (E). FIG. 2B depicts an illustration of system matrix slicing in voxel indices (rectangular cuboids with dotted edges) and virtual element indices (thick arcs with boundaries).

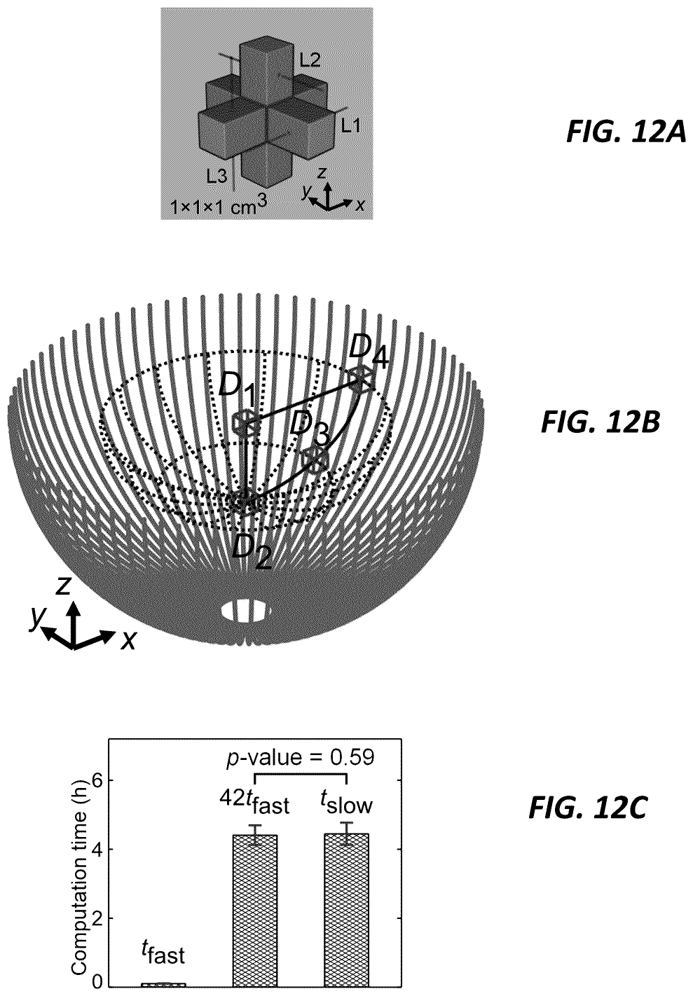

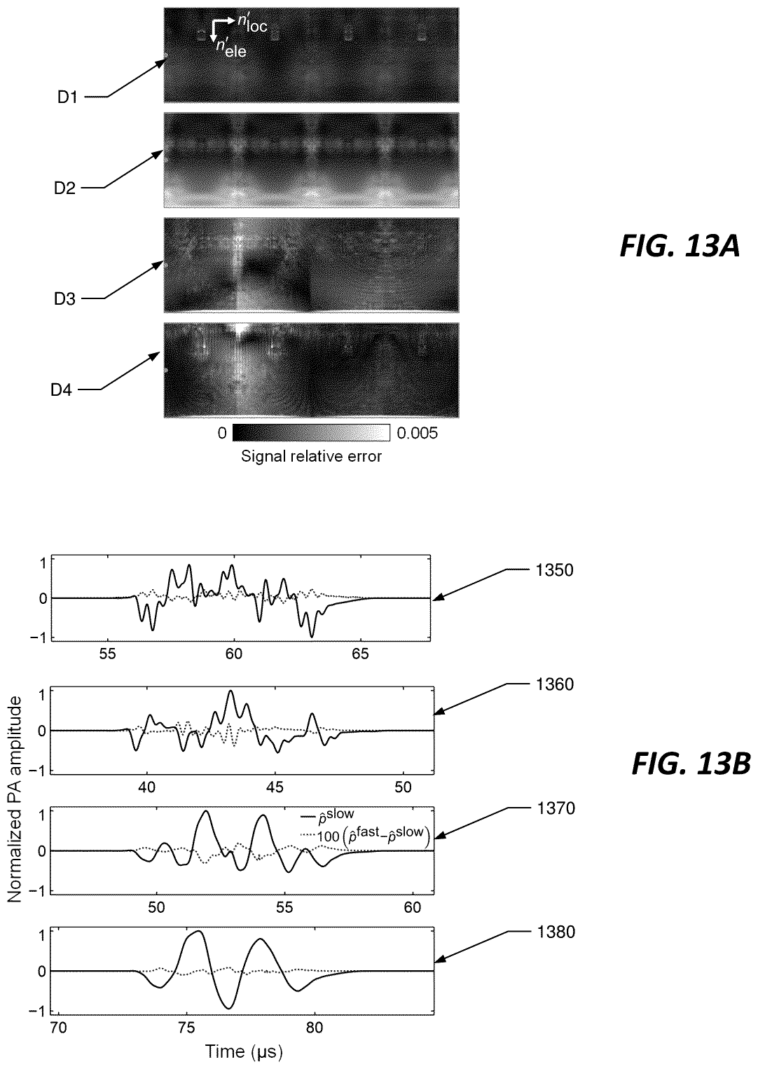

The original and compressed system matrices from a TISMC method may be described using a slow operator and a fast operator, respectively. The discretized operators are summarized in Eqns. 1 and 2. Through a forward simulation procedure, the compressed system matrix for the PACT system discussed above was shown to be approximately forty two (42) times as fast as the original system matrix with negligible signal-domain relative errors (maximum value of 0.005) as discussed in Section III (D) and shown in FIGS. 12A-C and FIGS. 13A-B. Further, for comparison, images were reconstructed using a nonnegativity-constrained iterative method described by Eqn. 33 with the compressed system matrix from signals simulated using the original system matrix. The images were observed to have negligible image-domain relative errors (maximum value of 0.007) as shown in FIG. 14 and discussed in Section III (E).

The implementation of the system matrix based on Eqn. 2 is not only efficient but also explicit, meaning subsets of the system matrix are directly accessible. For example, to obtain the signals from M′ voxels (enclosed in two rectangular cuboids with dotted edges in FIG. 2B) detected by N′ virtual elements (slender arcs between thick arcs in FIG. 2B), the relevant system matrix elements can be accessed directly and the computation in

$$\frac{{M}^{′} {N}^{′}}{MN}$$

of the time can be finished for M voxels and N elements.

According to various embodiments, a TISMC method performs piece-wise compression for each virtual sensor element of a tomographic system matrix. The piece-wise compression may only be performed once for a particular tomographic imaging system configuration as, for example, part of a calibration procedure. The piece-wise compression temporally shifts one or more of the responses to align arrival times and then group aligned responses for each sensor element. The TISMC method then performs an SVD procedure on the aligned responses to approximate the responses as linear combinations of a plurality of basis functions to obtain a compressed system matrix. The TISMC method may then be used to implement a fast forward operator to generate simulated virtual sensor signals. The fast forward operator method may then be used in an iterative reconstruction technique to reconstruct a tomographic image from detected signals.

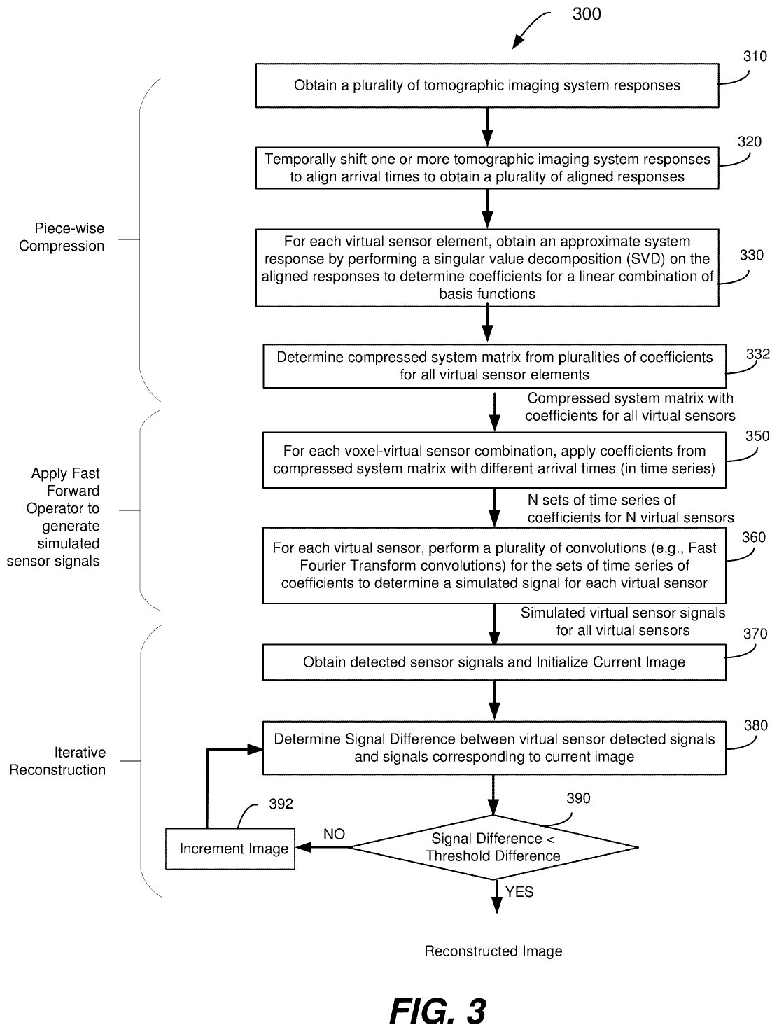

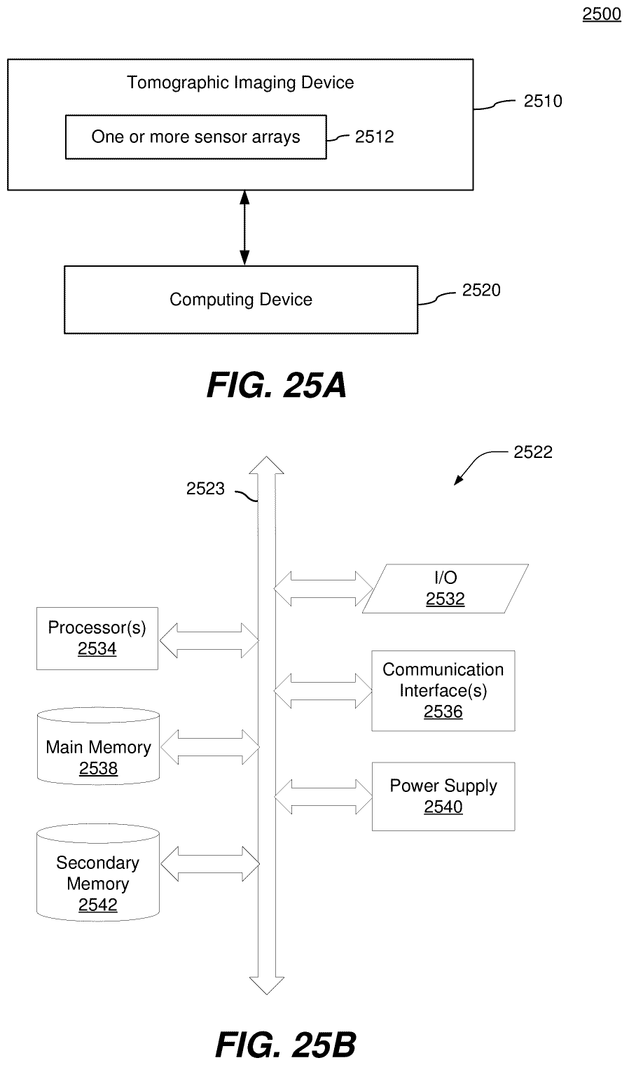

FIG. 3 depicts a flowchart illustrating operations of a TISMC method, according to various embodiments. One or more of the operations of the TISMC method may be performed by a computing device (e.g., computing device 2580 in FIG. 25A or computing device 2581 in FIG. 25B). The TISMC method includes a piece-wise compression process including operations 310, 320, and 330, a process that applies a fast forward operator to generate simulated sensor signals including operations 332 and 350, and a gradient descent iterative reconstruction process including operations 370, 380, 390, and 392. In another implementation, the TISMC method may employ an alternative iterative reconstruction process such as a FISTA procedure.

During an exemplary data acquisition phase of operation of a tomographic imaging system, sensor elements (e.g., transducers of one or more transducer arrays) are scanned to various detection locations at which signals are acquired from an object being imaged. As used herein, “virtual sensor elements” refer to the detection locations at which the sensor elements are located during signal acquisition. The detected signals may be stored to a buffer and/or recorded by one or more data acquisition systems of the tomographic imaging system. At operation 310, the TISMC method obtains a plurality of tomographic imaging system responses. The responses may be sensor signals detected during a calibration procedure when a calibration object is positioned for imaging in the tomographic imaging system. Alternatively, the responses may be simulated using the slow operator. The responses may be retrieved from memory or a buffer, or the signals may be received directly from the sensors of the tomographic imaging device.

At operation 320, the TISMC method temporally shifts one or more responses to align the arrival times of all responses. The arrival times of responses are determined by the voxel-sensor distances (i.e. distances between the voxel and the virtual sensor). As an example, in the illustrated example shown in FIG. 1B, the first responses 131, second response 132, . . . , MN response 133, are aligned to the same arrival time at dashed line 118.

At operation 330, the TISMC method performs a SVD procedure on the aligned responses to determine a plurality of coefficients for the linear combination of the basis functions that approximates the responses for each virtual sensor element. The plurality of basis functions may be obtained from the SVD procedure on the aligned responses. For example, the basis functions may be temporal singular vectors obtained from SVD procedure. An example of a plurality of basis functions are the first three temporal singular functions (e.g., three temporal singular functions 141, 142, and 143 in FIG. 1B). This SVD procedure is repeated for every virtual sensor element until a plurality of coefficients is determined for all the virtual sensor elements. At operation 332, the compressed system matrix is generated from the pluralities of coefficients 150 for all the virtual sensor elements.

At operation 350, for each voxel-virtual sensor combination, the TISMC method applies the coefficients from the compressed system matrix with the original arrival times to determine a time series of coefficients. For example, in the illustrated example shown in FIG. 2A, three time series of coefficients 201, 202, and 203 are obtained corresponding to the three temporal singular functions 141, 142, and 143 respectively. This operation is repeated for every voxel-virtual sensor combination until a time series of coefficients for all the voxel-virtual sensor combinations is determined.

At operation 360, for each virtual sensor, the TISMC method applies a plurality of convolutions (e.g., 3 convolutions for the three basis functions) to the sets of time series of coefficients to approximate a simulated signal for each virtual sensor. An example of a suitable convolution procedure that can be employed is convolution implemented by fast Fourier transform (FFT). This operation is repeated until a simulated signal is determined for a set of virtual sensors. In one embodiment, the operation is repeated until a simulated signal is determined for all virtual sensors. In another embodiment, the operation is repeated until a simulated signal is determined for every virtual sensor of a subset of all the virtual sensors.

At operation 370, the TISMC method obtains detected virtual sensor signals and initializes a current image. The detected sensor signals may be retrieved from memory or a buffer, or the signals may be received directly from the sensors of the tomographic imaging device. In one example, the voxels of the current image may be initialized with constant values.

At operation 380, the method determines a difference between the virtual sensor detected signals and the signals of the current image. At operation 390, if the difference between the virtual sensor detected signals and the signals of the current image is greater than, or equal to, a threshold difference, the image is incremented and the method returns to operation 380. An example of a threshold difference is 1% of the detected signals. If the difference between the virtual sensor detected signals and the signals of the current image is less than the threshold difference, a reconstructed image is output and the method ends.

### -Original Tomographic System Matrix and System Matrix Compressed by TISMC Method

Original and compressed tomographic imaging system matrices (compressed by a TISMC method according to an embodiment) can be described by a slow operator and a fast operator, respectively. The determination of the discretized operators is discussed in more detail in Section III (E) and is summarized below:

$${\hat{p}}_{n , l}^{slow} = {\sum }_{m = 1}^{M} {v}_{m} {h}_{n , m , l} {p}_{0 , m} , \qquad ( Eqn . 1 )$$

$$n = 1 , 2 , … , N , l = 1 , 2 , … , L ,$$

$$and$$

$$( Eqn . 2 )$$

$${\hat{p}}_{n , l}^{fast} = {\sum }_{k = 1}^{K} {η}_{k , l * l} {\sum }_{m = 1}^{M} {v}_{m} {\hat{h}}_{1 c 1 , k , n , m} {P}_{0 , m} \frac{1}{τ} [ \begin{matrix}( {t}_{{l}_{n , m} + 1} - \frac{‖ {r}_{m}^{′} - {r}_{n} ‖}{c} ) {δ}_{l , {l}_{n , m}} + \\ ( \frac{‖ {r}_{m}^{′} - {r}_{n} ‖}{c} - {t}_{{l}_{n , m} + 1} )\end{matrix} ] ,$$

$$n = 1 , 2 , … , N , l = 1 , 2 , … , L .$$

The time variable is discretized as t₁=t₁+(l−1)_{τ}, l=1, 2, . . . , L, where t₁ is the initial time, τ is the sampling step size, and L is the number of time points. In the operators, \hat{p}_{n,l} means the signal detected by the n-th virtual element at the time tₗ and is approximated by \hat{p}_{n,l}^{slow} and \hat{p}_{n,l}^{fast}, respectively; vₘ and p_{0,m} represents the volume and initial pressure of the m-th voxel, respectively; h_{n,m,l} is the n-th virtual element's response to the m-th voxel at tₗ; η_{k,l} is the k-th temporal singular function (obtained in SVD) evaluated at tₗ; K is the number of used singular value components; *l means the discrete convolution for the time index l (implemented in FFT); ĥ_{|c|,k,n,m} is the k-th spatial singular function's value depending on the m-th voxel's location relative to the n-th virtual element; r′ₘ and rₙ are the coordinates of the m-th voxel and n-th virtual element, respectively; l_{n,m} denotes the temporal index such that

$${t}_{{l}_{n , m}} \leq \frac{‖ {r}_{m}^{′} - {r}_{n} ‖}{c} < {t}_{{l}_{n , m} + 1} ,$$

and the following Kronecker delta function is used

$${δ}_{l , {l}^{′}} = \{ \begin{matrix}0 , l \neq {l}^{′} , \\ 1 , l = {l}^{′}\end{matrix} .$$

Although the numerical length of h_{n,m,l} (l=1, 2, . . . , L) is L, it has a smaller effective length, denoted as L′, for nonzero values. The symbols are summarized in Table 1 below. Numerical complexities of the slow and fast operators are O(NML′) and O(NMK)+O(N(Llog₂L)K), respectively. Here, the big O notations mean that there exist constants β₁, β₂, and β₃ (independent from NML′, NMK, and N (Llog₂L) K, respectively) such that the computations based on the slow and fast operators, respectively, can be done in times less than β₁NML′ and β₂NMK+β₃N (Llog₂L) K. The slow operator consists of only spatial integration; whereas, the fast operator consists of two layers of operations, and the terms β₂NMK and β₃N (Llog₂L) K are responsible for the inner layer spatial integration with ĥ_{|c|,k,n,m} and the outer layer temporal convolution with η_{k,l}, respectively. In numerical simulations with M=50×50×50, N=396×128, L=4096, L′=151, and K=3 discussed in Section III (D), it was observed that the fast operator based on Eqn. 2 has approximately forty two (42) times the speed of the slow operator based on Eqn. 1.

TABLE 1
Symbols for the forward operator and image reconstruction.

| | | |
|---|---|---|
| Symbol | Definition as applied to | Example Values for PACT system |
| | PACT system | |
| M (m) | The number of voxels in the | In functional imaging, M = 180 × 240 × |
| | image (index) | 160 for numerical simulations and M = |
| | | 250 × 250 × 200 for in vivo experiments; |
| | | in motion correction, M = 224 × 224 × |
| | | 72 for numerical simulations and M = |
| | | 300 × 300 × 150 for in vivo experiments |
| v_{m,1}, v_{m,2}, and | Voxel sizes along the x-axis, | (v_{m,1}, v_{m,2}, v_{m,3}) = (0.1, 0.1, 0.1) (mm) |
| v_{m,3} (m) | y-axis, and z-axis, | for functional imaging |
| | respectively | (v_{m,1}, v_{m,2}, v_{m,3}) = (0.2, 0.2, 0.2) (mm) |
| | | for motion correction |
| N_{loc} (n_{loc}) and | Rotating location numbers of | In functional imaging, 4N_{loc} = 396 for |
| 4N_{loc} (n_{loc}′) | the four-arc array (index) and | dense sampling and 4N_{loc} = |
| | single-arc array (index), | 76, 40, 28, 20, 16, 12 for sparse sampling; |
| | respectively | in motion correction, 4N_{loc} = 288 for |
| | | numerical simulations and 4N_{loc} = 396 |
| | | for in vivo experiments |
| Nₑₗₑ (nₑₗₑ) and $\frac{{N}_{ele}}{4} ( {n}_{ele}^{′} )$ | The numbers of transducer elements in the four-arc array (index) and single-arc array (index), respectively | $For numerical simlations , \frac{{N}_{ele}}{4} = 128 in$ $functional imagaing and \frac{{N}_{ele}}{4} = 72 in$ |
| | | motion correction; for in vivo |
| | | $experiments , \frac{{N}_{ele}}{4} = 256$ |
| a and b (m) | The length and width of each | a = 0.7 mm and b = 0.6 mm |
| | transducer element | |
| N (n) | The number of virtual | N = N_{loc}Nₑₗₑ |
| | elements in the 2D array | |
| | (index) | |
| τ (s) | The temporal sampling step | τ = 25 ns, which corresponds to a 40- |
| | size of each element | MHz sampling frequency |
| L | The temporal sampling | L = 4096 |
| | length of each element | |
| L′ | The effective length of the | L′ = 151 |
| | point source responses for | |
| | nonzero values | |
| Aₙ(m²) | The detection area of the n- | Aₙ = ab = 0.42 mm², n = 1, 2, ... , N |
| | th virtual element | |
| c (m · s⁻¹) | Speed of sound | c = 1.5 mm · μs⁻¹ in numerical |
| | | simulations, tuned for in vivo experiments |
| K | The number of used singular | K = 3 |
| | components (Eqns. S16 and | |
| | S27) | |

| | |
|---|---|
| vₘ (m³) | The volume of the m-th voxel: vₘ = v_{m,1}v_{m,2}v_{m,3} |
| r (m) | A location for ultrasonic signal detection |
| rₙ (m) | The center of the detection surface of the n-th virtual element |
| r′ (m) | A location in the image domain |
| rₘ′ (m) | The center of the m-th voxel in the image |
| t (s) | Time |
| tₗ (s) | The l-th sampling time |
| p₀(r′) (Pa) | The initial pressure at r′ |
| p(r, t) (Pa) | The pressure at location r and time t |
| h_{s,n} (r′, t) | The SIR of the n-th virtual element (Eqn. S3) |
| ĥ_{s,n}(r′, t) | The temporally shifted version of h_{s,n} (r', t) (Eqn. S8) |
| h_{e,n}(t) (s⁻¹) | The EIR of the n-th virtual element (Eqn. S5) |
| hₑ(t) (s⁻¹) | The EIR shared by all virtual elements |
| \hat{p}(rₙ, t) (Pa) | The pressure detected by the n-th virtual element at time t (Eqn. S6) |
| hₙ(r′, t) (m⁻³) | The point source response (from r′) received by the n-th virtual element |
| | (Eqn. S7) |
| ĥₙ(r', t) (m⁻³) | The temporally shifted version of hₙ (r′, t) (Eqn. S9) |
| Aₙ(m) | An orthonormal matrix formed by three vectors representing the local |
| | coordinates of the n-th virtual element (Eqn. S10) |
| r_{lcl}′(m) | A location in the local coordinate system of a virtual element |
| r_{lcl,m}_{_{lcl}}′ (m) | The m_{lcl}-th location of interest in a virtual element’s local coordinate |
| | system |
| ĥ_{s,lcl}(r_{lcl}′, t) | The temporally shifted SIR of a virtual element expressed in its local |
| | coordinates (Eqn. S12) |
| ĥ_{lcl}(n_{lcl}′, t) (m⁻³) | The temporally shifted point source response of a virtual element |
| | expressed in its local coordinates (Eqn. S13) |
| ĥ_{lcl,k} (r_{lcl}′) (m⁻³), | The k-th spatial singular function and the k-th temporal singular function, |
| and ηₖ(t) | respectively, in the SVD of ĥ_{lcl}(r_{lcl}′, t) (Eqn. S16) |
| ĥ_{lcl,m}_{_{lcl}}_{,l}(m⁻³) | The value of ĥ_{lcl,k}(r_{lcl,m}_{_{lcl}}′, tₗ) obtained through Eqn. S25 |
| h̅_{lcl,k,m}_{_{lcl}} (m⁻³) | The value of ĥ_{lcl,k}(r_{lcl,m}_{_{lcl}}′) obtained through Eqn. S27 |
| ĥ_{lcl,k,n,m} (m⁻³) | The value of ĥ_{lcl,k}((rₘ′ − rₙ)Aₙ) obtained through interpolation of |
| | h̅_{lcl,k,m}_{_{lcl}} |
| η_{k,l} | The value of ηₖ (tₗ) obtained through Eqn. S27 |
| h_{n,m,l}(m⁻³) | The value of hₙ(rₘ′, tₗ) |
| p_{0,m}(Pa) | The value of p₀ (rₘ′) |
| \hat{p}_{n,1}(Pa) | The value of \hat{p}(rₙ,tₗ) |
| \hat{p}_{n,l}^{slow}(Pa) | An approximation of \hat{p}_{n,l} using the slow operator defined in Eqn. S26 |
| \hat{p}_{n,l}^{fast} (Pa) | An approximation of \hat{p}_{n,l} using the fast operator defined in Eqn. S30 |
| p₀ (Pa) | A vector formed by p_{0,m}, m = 1, 2, ... , M |
| H | A system matrix formed by the coefficients in Eqn. S26 or Eqn. S30 |
| \hat{p}(Pa) | A vector formed by \hat{p}_{n,l}, n = 1, 2, ... , N, l = 1, 2, ... , L |
| \hat{p}₀(Pa) | A vector formed by the reconstructed voxel values |
| λ(m) | The regularization parameter in the regularized optimization (Eqn. S34) |
| γ_{c} (Pa⁻¹) | The system-specific measurement calibration factor (ignored in practice) |
| \|p₀\|_{TV} (Pa · m⁻¹) | The TV norm of p₀ defined in Egn. S35 |

The unit of an array represents the unit of its elements.
B. Hybrid Methods for Fast Functional Imaging with Sparse Sampling in Tomography

Certain embodiments pertain to a hybrid method for functional imaging with sparse sampling in tomography that employs both an iterative reconstruction technique (e.g., FISTA or gradient descent method) and a universal back-projection (UBP) method. An example of an iterative reconstruction procedure is the iterative gradient descent algorithm. Another example of suitable an iterative reconstruction procedure is the fast iterative shrinkage-thresholding algorithm FISTA, which can be found in Beck, A. & Teboulle, M. A, “fast iterative shrinkage-thresholding algorithm for linear inverse problems,” SIAM J. Imaging Sci. 2, 183-202 (2009), which is hereby incorporated by reference in its entirety. An example of a UBP method can be found in Xu, M. & Wang, L. V., “Universal back-projection algorithm for photoacoustic computed tomography,” Phys. Rev. E 71, 016706 (2005), which is hereby incorporated by reference in its entirety.

-Examples of Hybrid methods

During an exemplary data acquisition phase of operation of a tomographic imaging system, sensor elements (e.g., transducers of one or more transducer arrays) are scanned (by rotating/translating) to various detection locations at which signals are acquired from an object being imaged. As used herein, “virtual sensor elements” refer to the detection locations at which the (physical) sensor elements are located during signal acquisition. In one example, data acquisition may involve a dense sampling operation and a fast functional imaging operation. During the dense sampling operation, signals are acquired from an object with sensor elements at enough detection locations (N) for dense sampling. An example of a suitable number of dense sampling locations, N, is 256*400. During the fast functional imaging operation, multiple sets of signals are acquired from the object with sensor elements at a smaller number of locations (N′) for sparse sampling for fast functional imaging. An example of a suitable number of sparse sampling locations, N′, is 256*12. It is assumed that the object does not move as a whole during functional imaging. In various embodiments, the hybrid method obtains the signals from dense sampling and the signals from sparse sampling for fast functional imaging. The signals may be recorded by the tomographic imaging device (e.g., by data acquisition system(s)) and may be retrieved from memory or a buffer, or the signals may be received directly from the tomographic imaging device.

According to certain embodiments, the hybrid method reconstructs a densely sampled prior image from the signals from dense sampling using an iterative reconstruction process and incorporates the densely sampled prior image into a dense-sampling system matrix and a sparse-sampling system matrix. The hybrid method performs iterative reconstruction of a smooth modulated image from a set of functional signals. The smooth modulation image is upsampled and multiplied by the densely sampled prior image to obtain a modulated prior image. The dense-sampling system matrix is used to obtain a dense forward solution from the modulated prior image and a modulated UBP image is reconstructed from the dense forward solution using UBP. The sparse-sampling system matrix is used to obtain a sparsely sampled forward solution from the modulated prior image and a residual image is reconstructed from a difference between the sparse sampled signals and the sparsely sampled forward solution using UBP. A functional hybrid image may then be obtained by summing the modulated UBP image and the residual image. This process may be repeated for each of plurality of sets of functional signals to generate a plurality of hybrid functional images.

In various embodiments, a hybrid method reconstructs a modulated UBP image and a residual image from a set of sparsely sampled signals. In these embodiments, a UBP method may be employed to perform image reconstruction. The hybrid method may also reconstruct the densely sampled prior image from the signals from dense sampling using an iterative reconstruction technique such as, e.g., FISTA or a gradient descent technique.

For the image reconstruction examples discussed below, the densely sampled signals and a set of sparsely sampled signals may be denoted as \hat{p}_{NL×1} and \hat{p}_{N′L×1}^{s}, respectively. In these examples, an image \hat{p}_{0,M×1} may be obtained from the densely sampled signals, \hat{p}_{NL×1}, by solving the regularized optimization problem:

$$( Eqn . 3 )$$

$${\hat{p}}_{0 , M \times 1} = {arg min}_{{p}_{0 , M \times 1} ∈ {ℝ}^{M} , {p}_{0 , M \times 1} \geq 0} {γ}_{c} {‖ {H}_{NL \times M} {p}_{0 , M \times 1} - {\hat{p}}_{NL \times 1} ‖}^{2} + λ {\vert {p}_{0 , M \times 1} \vert}_{TV} .$$

Where: γ_{c} represents the system-specific measurement calibration factor, meaning γ_{c}\hat{P}_{NL×1} is the reading of the data acquisition system, H_{NL×M} is the dense-sampling system matrix; |p_{0,M×1}|_{TV} denotes p_{0,M×1}'s total variation (TV) norm, defined in Eqn. S35, and λ means the regularization parameter.

To obtain a tomographic image from \hat{P}_{N′L×1}^{s} directly, \hat{P}_{N′L×1}^{s} could be used and the sparse-sampling system matrix H_{N′L×M}^{s} could be used to replace \hat{P}_{N′L×1}^{s} and H_{NL×M}, respectively, in Eqn. 3 and solve it. However, the nonlinearity introduced by the nonnegativity constraint and TV regularization may disrupt the functional signals. Advantageously, to overcome this, the hybrid method, according to various embodiments, maintains linearity under sparse sampling of the functional signals by treating the densely sampled image, \hat{P}_{0,M×1}, as a prior image during image reconstruction from sparsely sampled signals.

In various examples, a smooth modulation is applied to the prior densely sampled image to extract the dominant sources of aliasing artifacts. This may be done, for example, by solving the following optimization problem using an iterative reconstruction algorithm (e.g., shrinkage-thresholding algorithm (FISTA)) with a constant step size:

$$( Eqn . 4 )$$

$${\hat{μ}}_{0 , {M}^{′}} \times 1 = {arg min}_{{P}_{0 , {M}^{′}} ∈ {ℝ}^{{M}^{′}}} {‖ {H}_{{N}^{′} L \times M}^{s} ( {\hat{p}}_{0 , M \times 1} ⊙ ( {U}_{M \times {M}^{′}} {μ}_{0 , {M}^{′} \times 1} ) ) - {\hat{p}}_{{N}^{′} L \times 1}^{s} ‖}^{2}$$

Where μ_{0,M′×1} is a modulation image in the form of a column vector of size M′=M′₁ M′₂ M′₃ (<<M), the symbol ⊙ denotes the element-wise product, and U_{M×M′} is an upsampling operator transferring μ_{0,M′×1} to a smooth modulation image in the column-vector form of size M.

In some cases, to implement U_{M×M′}, the vector μ_{0,M′×1} may be reshaped into a 3D array μ_{0,M′}_{₁}_{×M′}_{₂}_{×M′}_{₃}, a trilinear interpolation is applied to the 3D array to obtain μ_{0,M}_{₁}_{×M}_{₂}_{×M}_{₃}, and it is flattened to obtain U_{M×M′}μ_{0,M′×1}. The array μ_{0,M}_{₁}_{×M}_{₂}_{×M}_{₃} is a smooth array determined only by M′ independent values. Thus, the expression \hat{P}_{0,M×1} ⊙(U_{M×M′}μ_{0,M′×1}) represents a smooth modulation of the prior image \hat{P}_{0,M×1}. By solving the optimization problem in Eqn. 4, \hat{μ}_{0,M′×1} may be obtained and the dominant sources causing aliasing artifacts is represented by \hat{P}_{0,M×1} ⊙(U_{M×M′}\hat{μ}_{0,M′×1}), which correspond to the dominant signals causing aliasing artifacts H_{N′L×M}^{s} (\hat{P}_{0,M×1} ⊙(U_{M×M′}\hat{μ}_{0,M′×1})). The implementation of the forward operator based on Eqn. S30 allows for efficient slicing of the system matrix, such as the matrix H_{N′L×M}^{s}, a slicing with respect to transducer element indices. Also, for a given set of parameters in the reconstruction process (e.g. FISTA), the solution to Eqn. 4, \hat{μ}_{0,M′×1}, may depend linearly on \hat{P}_{N′L×1}^{s}.

In these example, the hybrid method applies the dense-sampling system matrix H_{NL×M} to \hat{P}_{0,M×1} ⊙(U_{M×M′}\hat{μ}_{0,M′×1}) to simulate the densely sampled signals H_{NL×M} (\hat{P}_{0,M×1}⊙(U_{M×M′}\hat{μ}_{0,M′×1})), from which the UBP method can be used to reconstruct a dense forward image, denoted as \hat{P}₀^{m}. The hybrid method may further remove the aliasing artifacts caused by signals from \hat{P}_{0,M×1} ⊙(U_{M×M′}\hat{μ}_{0,M′×1}) by subtracting these signals from P_{N′L×1}^{s}, to obtain the residual signals P_{N′L×1}^{s}−H_{N′L×M}^{s}(\hat{P}_{0,M×1} ⊙(U_{M×M′}\hat{μ}_{0,M′×1} and use them with the UBP method to reconstruct a residual image, denoted as \hat{P}₀^{r}.

Due to the linearity of the UBP method, both dense forward and residual images, \hat{P}₀^{m} and \hat{P}₀^{r}, respectively, are linearly dependent on \hat{μ}_{0,M′×1} and thus on \hat{P}_{N′L×1}^{s}. The hybrid method combines these two images to obtain the final hybrid functional image:

$${\hat{p}}_{0}^{h} = {\hat{p}}_{0}^{m} + {\hat{p}}_{0}^{r} , \qquad ( Eqn . 5 )$$

for \hat{P}_{N′L×1}s. The hybrid method repeats the process for other sets of sparsely sampled signals (raw data) to obtain the functional images for further functional signal extraction. Additional symbols referenced for fast functional imaging are shown in Table 2.

According to various embodiments, a hybrid method reconstructs a densely sampled prior image using an iterative construction procedure such as, e.g., FISTA. The hybrid method reconstructs a smooth modulation image from each set of one or more functional signals to generate a smooth modulation image. The smooth modulation image is upsampled and multiplied by the densely sampled prior image. A dense-sampling matrix is applied to the modulated prior image to obtain a dense forward solution from which a modulated UBP image is reconstructed using UBP. A sparse-sampling matrix is applied to the modulated prior image to obtain a sparsely sampled forward solution. A residual image is reconstructed using UBP from the difference between the detected sparse sampled signals and the sparsely sampled forward solution (residual data). A functional image is formed by the sum of the dense image and the residual data.

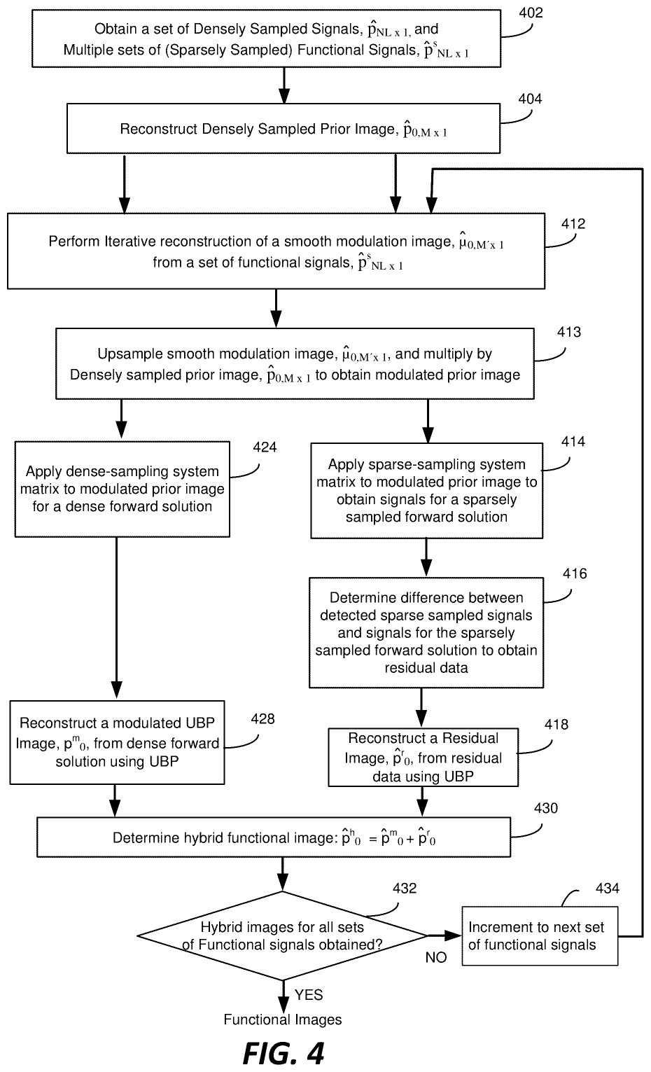

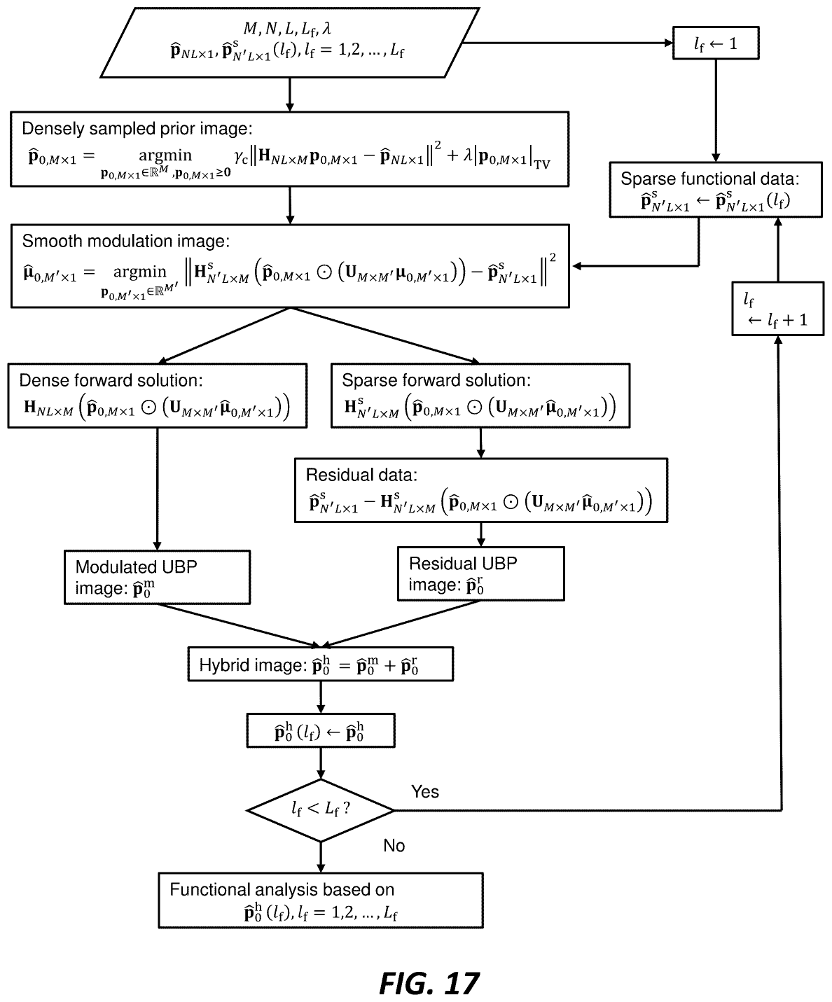

An example of a workflow of operations of an example of a hybrid method is shown in FIG. 17. FIG. 4 depicts a flowchart illustrating operations of a hybrid method, according to various embodiments.

During an exemplary data acquisition phase of operation of a tomographic imaging system, sensor elements are scanned (by rotating/translating) to various detection locations at which signals are acquired from an object being imaged. During the dense sampling operation, signals are acquired from an object with sensor elements at enough detection locations (N) for dense sampling. An example of a suitable number of dense sampling locations, N, 256*400. During the fast functional imaging operation, multiple sets of sparsely sampled signals are acquired from the object with sensor elements at a smaller number of locations (N′) for sparse sampling for fast functional imaging. An example of a suitable number of sparse sampling locations, N′, is 256*12.

At operation 402, a set of densely sampled signals and multiple sets of sparsely sampled signals are acquired. The signals may be retrieved from memory or a buffer, or the signals may be received directly from the sensors of the tomographic imaging device.

At operation 404, the hybrid method reconstructs a densely sampled prior image using an iterative reconstruction technique such as, e.g., FISTA or a residual gradient method. In one implementation, the image is reconstructed by solving Eqn. 3.

At operation 412, the hybrid method uses an iterative reconstruction technique to reconstruct a smooth modulation image from one of the sets of functional signals. In one implementation, the image is reconstructed by solving Eqn. 4.

At operation 413, the hybrid method upsamples the modulation image and multiples by the uses an iterative reconstruction technique to reconstruct a smooth modulation image from one of the sets of functional signals.

At operation 414, the hybrid method applies a sparse-sampling system matrix to the modulated prior image to obtain signals for a sparsely sampled forward solution.

At operation 416, the hybrid method determines a difference between the detected sparsely sampled signals and the sparsely sampled forward solution to obtain the residual data.

At operation 418, the hybrid method reconstructs a residual image from the residual data using UBP.

At operation 424, the hybrid method applies a dense-sampling system matrix to the modulated prior image to obtain signals for a densely sampled solution.

At operation 428, the hybrid method reconstructs a modulated UBP image from the dense forward solution using UBP.

At operation 430, the hybrid method determines a hybrid functional image from a sum of the modulated UBP image and the residual image.

At operation 432, the hybrid method determines whether hybrid functional images have been obtained for all the sets of functional signals. If all functional images have not been obtained, the method increments to the next set of functional signals and returns to operation 412 to uses an iterative reconstruction technique to reconstruct a smooth modulation image from the next set of functional signals (operation 434). If all functional images have been obtained, the method ends.

-Demonstration of Using an Example of a Hybrid Method as Implemented with a PACT System

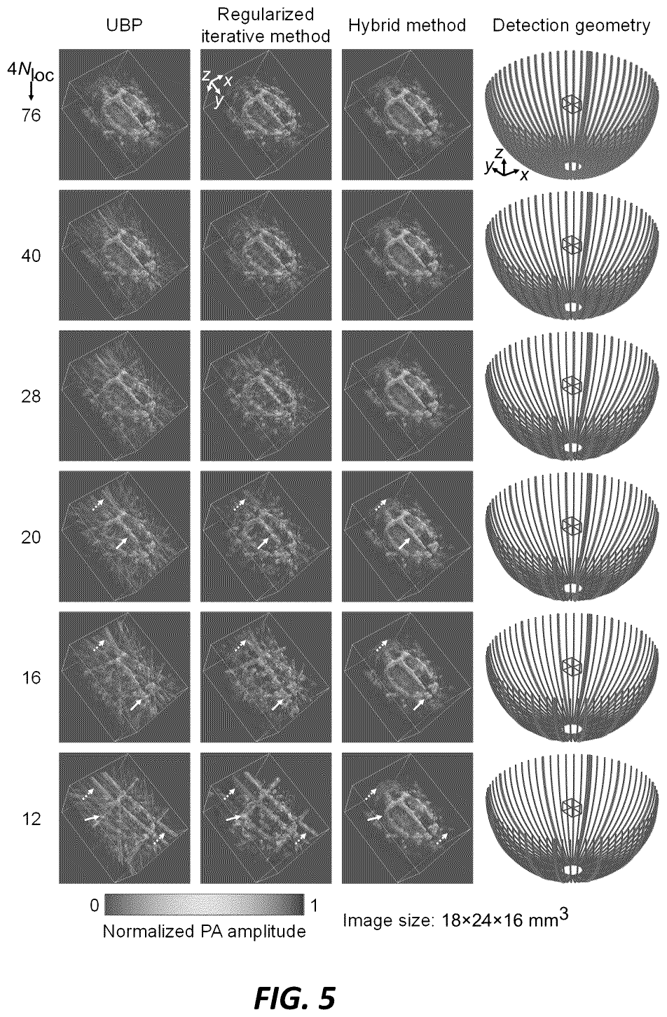

A 3D PACT system with four arc transducer arrays (with 128 transducer elements in each arc array) was used to demonstrate the performance of an example of a hybrid method by using the hybrid method to reconstruct images from signals recorded by the 3D PACT system of a numerical phantom. During six (6) data acquisition runs, different virtual detection geometry was used with six different numbers of rotating arc locations: 4N_{loc}=76, 40, 28, 20, 16, and 12. During the data acquisition runs, the four arc transducer arrays recorded signals at different numbers of rotating arc locations: 4N_{loc}=76, 40, 28, 20, 16, and 12. Here, N_{loc} is the number of rotating locations of the four-arc transducer array in a virtual transducer array. Images of the numerical phantom reconstructed using UBP, the regularized iterative method based on Eqn. S34, and the hybrid method based on Eqn. 5 with a prior image obtained by performing a smooth modulation to the numerical phantom are shown in the first three columns in FIG. 5.

FIG. 5 depicts images reconstructed by UBP, regularized iterative method, and hybrid method (first three columns) from signals detected at sparsely distributed elements (thicker curves in the fourth column) for 4N_{loc}=76, 40, 28, 20, 16, 12, according to an embodiment. The detection geometry of the virtual transducer arrays used to detect signals are shown in the images of the fourth column in FIG. 5. Examples of maintained features and suppressed artifacts are indicated by white-solid and white-dotted arrows, respectively.

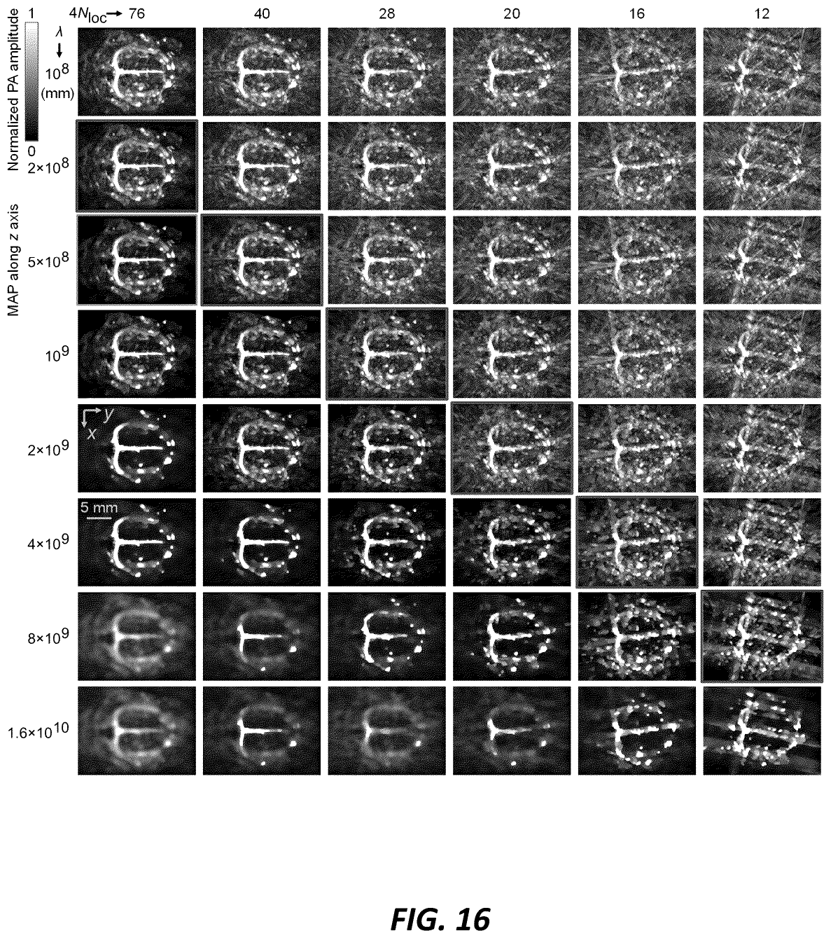

For comparison to the hybrid method, a regularized iterative reconstruction process was used to determine functional images with sparse sampling. An example of a selection of a regularization parameter is discussed in Section III (A). FIG. 16 illustrates mouse brain images reconstructed with a regularized iterative reconstruction process from sparse sampling signals for 4N_{loc}=76, 40, 28, 20, 16, and 12.

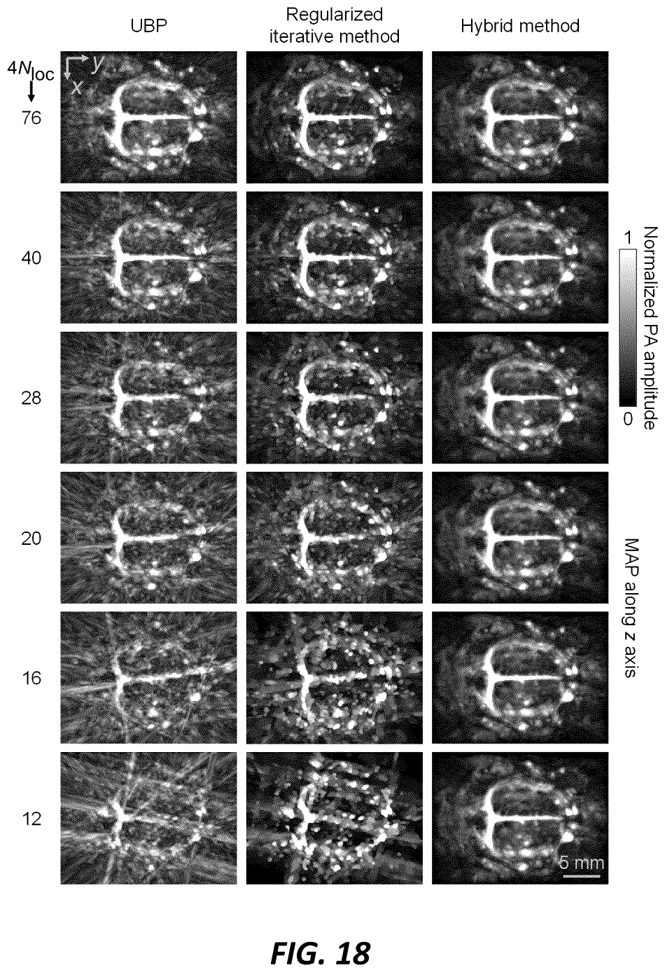

FIG. 18 depicts maximum amplitude projections (MAPs) of these mouse brain images along the z-axis using UBP, the regularize iterative method, and a hybrid method, according to an embodiment. It is seen in these MAPs that as 4N_{loc} decreases from 76 to 12, the artifacts in the images reconstructed using UBP become more abundant. The regularized iterative method mitigates the relatively weak artifacts in all images but failed to suppress the strong artifacts such as in the images with 4M_{loc}=16, 12. In comparison, with the help of the prior (densely sampled) image, the hybrid method significantly mitigates the artifacts. Quantitatively, for each method, the structural similarity index measures (SSIMs) were calculated between the images with 4N_{loc}=40, 28, 20, 16, and 12 and that with 4N_{loc}=76.

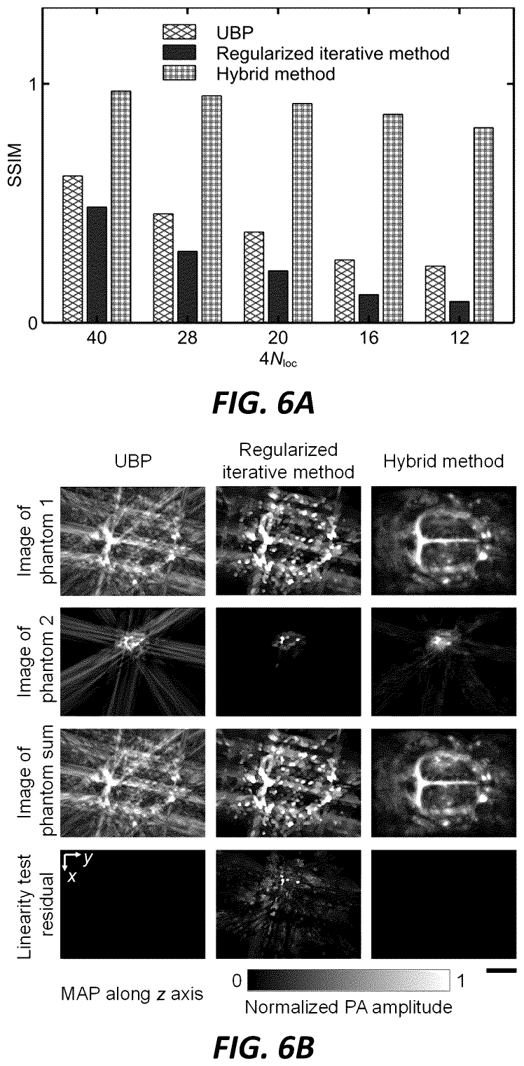

FIG. 6A depicts structural similarity index measures (SSIMs) between the reconstructed images in FIG. 5 with 4N_{loc}=40, 28, 20, 16, and 12 and those with 4N_{loc}=76 for the three methods: UBP, regularized iterative method, and hybrid method. As shown, the hybrid method performs the best in mitigating artifacts and maintaining true features.

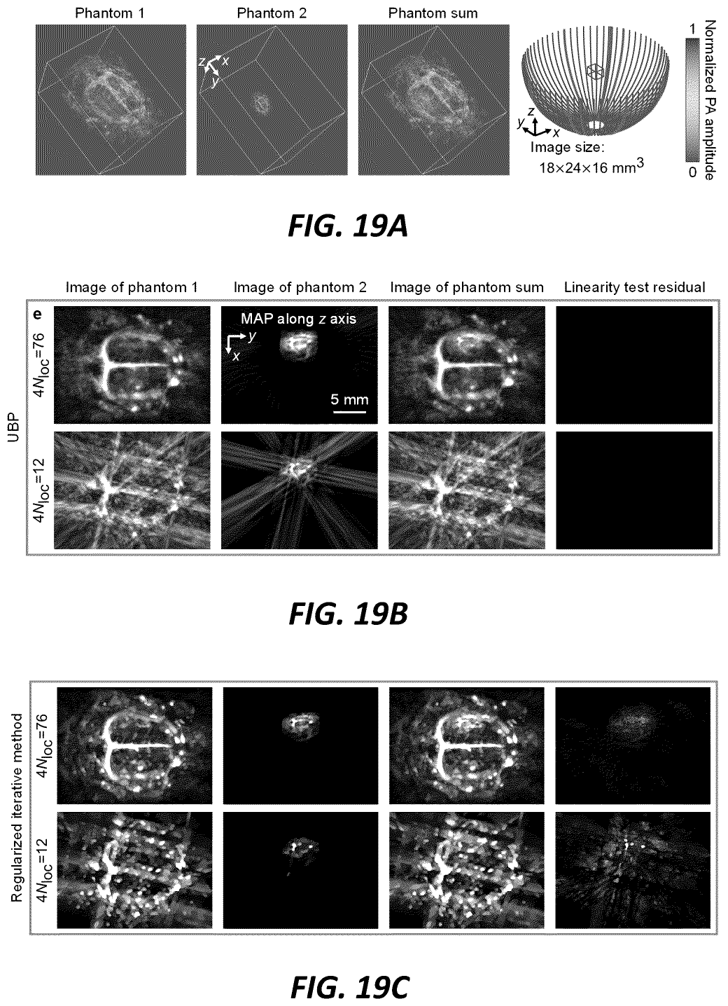

Additionally, the linearity was tested for each method by reconstructing two numerical phantoms and their summation as shown in FIG. 19A. MAPs of the reconstructed images and the linearity test residuals (image of the summation subtracted by images of the two phantoms) of the three methods with 4N_{loc}=76, 12 are shown in FIGS. 19A-C. From the linearity test residuals, the hybrid method appears to maintain linearity while mitigating artifacts. MAPs in the linearity tests with 4N_{loc}=12 are shown in FIG. 6B.

In another test of the hybrid method using a prior image with dot artifacts shown in FIG. 19B, the linearity is still valid although artifacts appear in the reconstructed images as shown in FIG. 19C. In summary, UBP and the hybrid method are linear, but the regularized iterative method is nonlinear. In the hybrid method, the prior image quality does not affect the linearity but affects the reconstructed image quality. FIG. 6B depicts linearity tests of the three methods: UBP, regularized iterative method, and hybrid method for 4N_{loc}=12. Scale bar, 5 mm.

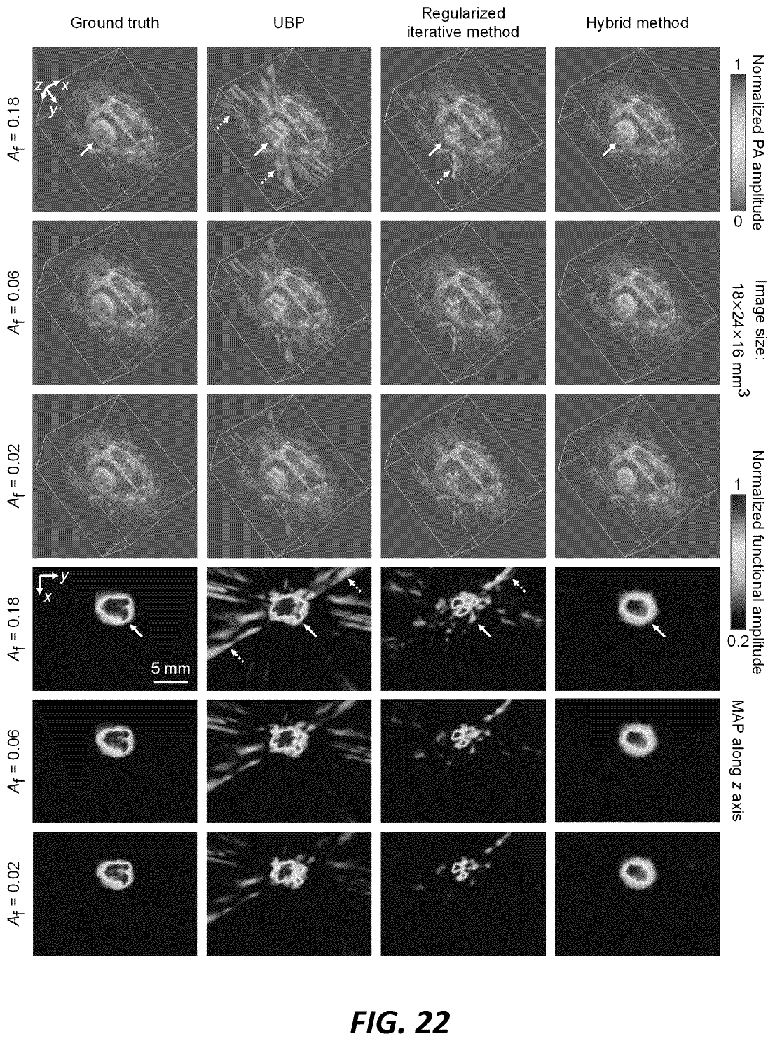

Due to use of a prior image, the practical value of a hybrid method of certain examples mainly lies in fast functional imaging with sparse sampling, although other practical values are contemplated such as mitigating aliasing artifacts by numerically generating a prior image from an initial reconstruction from another method and use the prior image for a second reconstruction using the hybrid method. Numerical simulations of functional imaging using a hybrid method with 4N_{loc}=12 are discussed in Section III (G). After reconstructing images from simulated signals using the three methods, functional signals were extracted from them using a method based on a regularized correlation based on Eqn. 7 to form functional images. As shown in FIG. 22, artifacts in the UBP-reconstructed images cause artifacts in the functional images, the regularized iterative method mitigates artifacts in functional images but also compromises the true functional region, and the hybrid has the best performance.

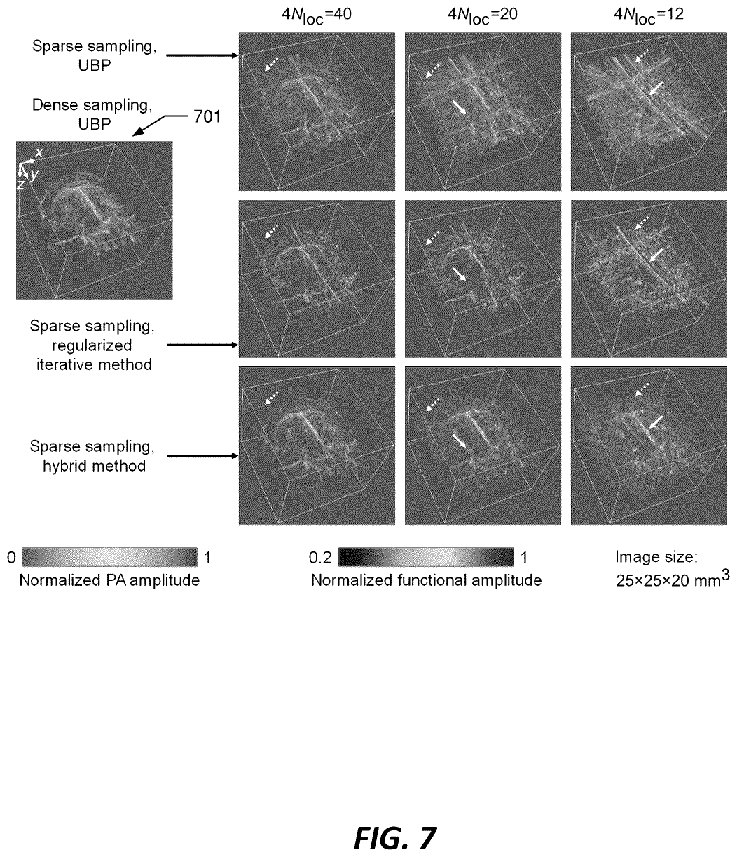

As another demonstration, UBP, the regularized iterative method, and the hybrid method were applied to mouse brain functional imaging in vivo using the four-arc PACT system. A prior image of the mouse brain through dense sampling (4N_{loc}=396), and then electrically stimulate was applied to the mouse's right front paw and signals were continuously acquire from the mouse brain through sparse sampling (4M_{loc}=76, 2 s per image) by rotating transducer arrays. Subsets of the sparsely sampled signals (4N_{loc}=40, 20, 12) were used to demonstrate the performance of the hybrid method. For one set of sparsely sampled signals, the images reconstructed using UBP, the regularized iterative method, and the hybrid method for 4N_{loc}=40, 20, and 12 are shown in FIG. 7.

FIG. 7 depicts a densely sampled image 701 of a mouse brain reconstructed by UBP with N_{loc}=396 and sparsely sampled images of the mouse brain reconstructed using UBP (first row), the regularized iterative method (second row), and the hybrid method (third row) according to an implementation, respectively, for 4N_{loc}=40, 20, 12. Examples of suppressed artifacts and maintained features are indicated by white-dotted and white-solid arrows, respectively.

Based on the results shown in FIG. 7, the regularized iterative method mitigates the artifacts (e.g., those indicated by white-dotted arrows) but compromises low-amplitude features (e.g., those indicated by white-solid arrows for 4N_{loc}=20). In contrast, the hybrid method of this example maintains low-amplitude features while substantially mitigating the artifacts, resulting in images more similar to the densely sampled image.

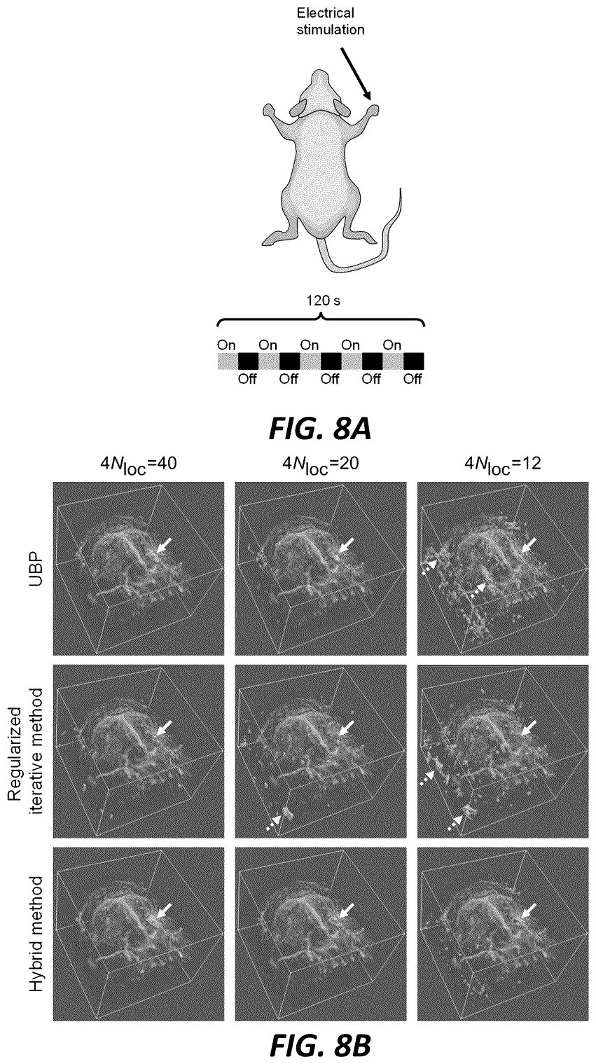

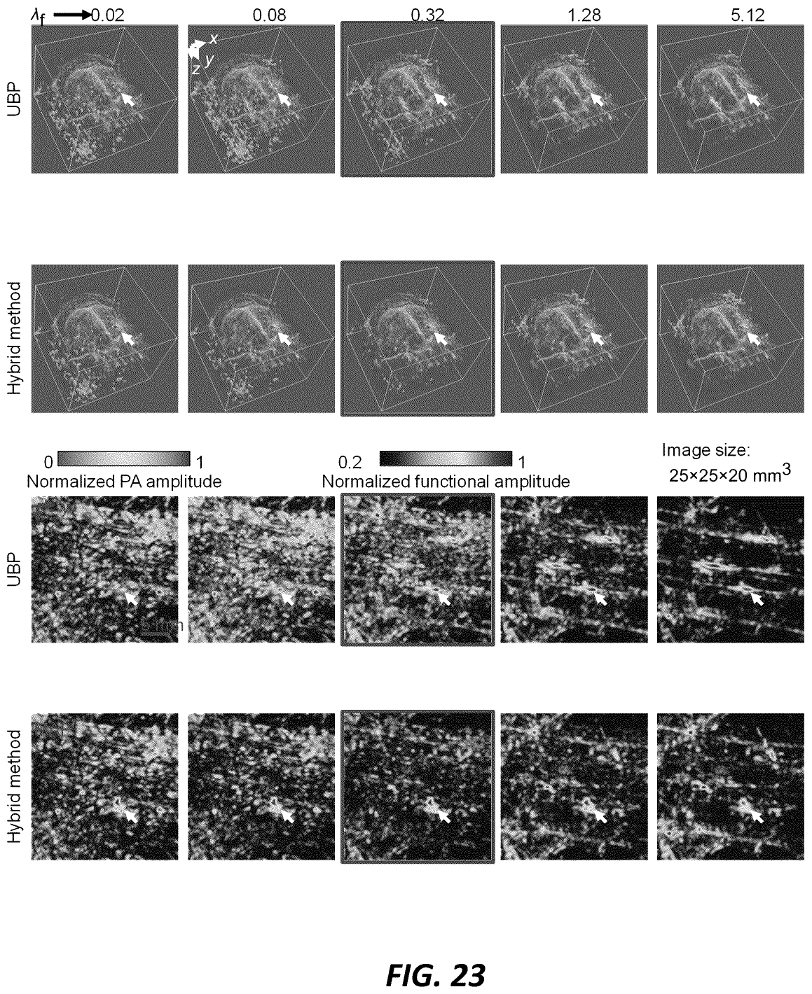

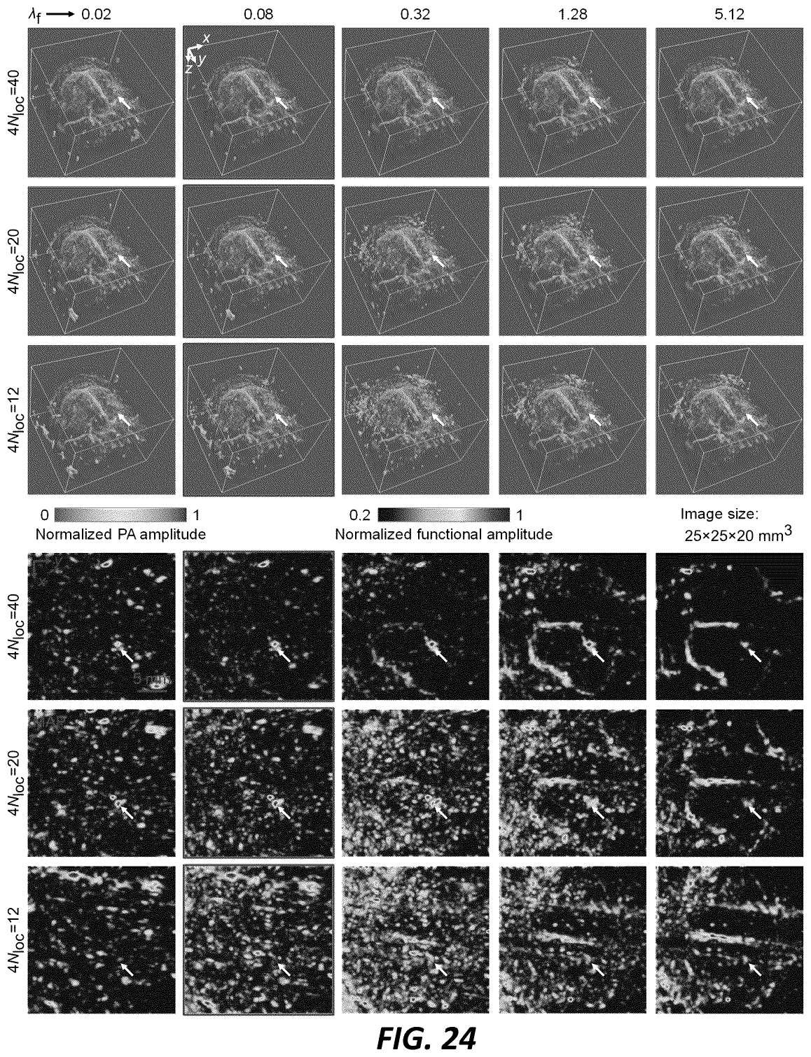

In this demonstration, electrical stimulation of the mouse's right front paw occurred in five cycles, each with 12-s stimulation on and 12-s off, as shown in FIG. 8A. To find the best regularization parameter (λ_{f}) in the regularized correlation in Eqn. 7, functional images were obtained from the images reconstructed through the three methods for 4N_{loc}=40, 20, 12 using λ_{f}=0.02, 0.08, 0.32, 1.28, 5.12. For UBP and the hybrid method, λ_{f}=0.32 is the best choice to maintain the true functional region and suppress false positive regions, and the functional images for 4N_{loc}=12 are shown in FIG. 23. For the regularized iterative method, the images for 4N_{loc}=40, 20, 12 were compared in FIG. 24 and it was determined that λ_{f}=0.08 is the best choice. The obtained functional images with best values of λ_{f} in FIG. 8B.

FIG. 8B depicts functional images obtained from the images reconstructed using UBP (first row, λ_{f}=0.32), the regularized iterative method (second row, λ_{f}=0.08), and the hybrid method according to an embodiment (third row, λ_{f}=0.32), respectively, for 4N_{loc}=40, 20, 12. The true functional regions in all images are indicated by white-solid arrows, and examples of false positive regions are indicated by white-dotted arrows.

Results from UBP and the hybrid method match well for 4N_{loc}=40. The hybrid method is slightly (significantly) better than UBP for 4N_{loc}=20 (4N_{loc}=12). Due to the violation of linearity, the regularized iterative method compromises the true functional region: leading to its shrinkage for 4N_{loc}=40, 20 and its decimation altogether for 4N_{loc}=12. In summary, the hybrid method enables fast functional imaging with highly sparse sampling.

TABLE 2
Additional symbols for fast functional imaging with sparse sampling.

| Symbol | Definition | Value |
|---|---|---|
| L_{f} (l_{f}) | The number of sparsely | L_{f} = 36 for numerical simulations, and |
| | sampled images in | L_{f} = 60 for in vivo experiments |
| | functional imaging (index) | |
| α_{f, l}_{_{f}} | A functional signal profile | Defined by (Eqn. S37) for numerical |
| | | simulations, and obtained from UBP- |
| | | reconstructed images with 4N_{loc} = 76 for |
| | | in vivo experiments |
| A_{f} | The functional amplitude | A_{f} = 0.18, 0.06, 0.02 |
| | in (Eqn. S37) | |

| | |
|---|---|
| \hat{p}_{NL×1} (Pa) | Pressures detected by N densely distributed virtual elements at L time |
| | points |
| \hat{p}_{N′ L×1}^{s} (Pa) | Pressures detected by N′ sparsely distributed virtual elements at L |
| | time points |
| p_{0, M×1} (Pa) | Values of the M voxels |
| H_{NL×M} | A dense-sampling system matrix transferring p_{0, M×1} to \hat{p}_{NL×1} |
| H_{N′ L×M}^{s} | A sparse-sampling system matrix transferring p_{0, M×1} to \hat{p}_{N′ L×1}^{s} |
| \hat{p}_{0, M×1} (Pa) | Reconstructed values of the M voxels (Eqn. 3) |
| μ_{0, M′×1} | The column-vector form of a modulation image of size M′ (<<M) |
| U_{M×M′} | An upsampling operator transferring μ_{0, M′×1} to a smooth modulation |
| | image of size M in the column-vector form |
| \hat{μ}_{0, M′×1} | The modulation image reconstructed for \hat{p}_{N′ L×1}^{s} in (Eqn. 4) |
| \hat{p}₀^{m} (Pa) | An image reconstructed by UBP from the signals of modulated prior |
| | image H_{NL×M} (\hat{p}_{0, M×1} ⊙ (U_{M×M′} \hat{μ}_{0, M′×1})): the modulated UBP |
| | image |
| \hat{p}₀^{r} (Pa) | An image reconstructed by UBP from the residual signals \hat{p}_{N′ L×1}^{s} − |
| | H_{N′ L×M}^{s} (\hat{p}_{0, M×1} ⊙ (U_{M×M′} \hat{μ}_{0, M′×1})): the residual UBP image |
| \hat{p}₀^{h} (Pa) | The hybrid image defined as \hat{p}₀^{h} = \hat{p}₀^{m} + \hat{p}₀^{r} (Eqn. 5) |
| \hat{p}_{0, l}_{_{f}} (Pa) | The l_{f}-th sparsely sampled image |
| p_{0, l}_{_{f}} (Pa) | The l_{f}-th numerical phantom in functional simulation obtained by (Eqn. |
| | S36) |
| p_{0, b} (Pa) | A background numerical phantom obtained in imaging with dense |
| | sampling |
| p_{0, f} (Pa) | A functional numerical phantom obtained from smoothing, |
| | downsampling, and zero padding p_{0, b} |
| α_{b, l}_{_{f}} | The background modulation factor for the l_{f}-th numerical phantom |
| \hat{p}_{0, l}_{_{f}}_{, m} (Pa) | The value of the m-th voxel in \hat{p}_{0, l}_{_{f}} |
| [symbol] | The Pearson correlation coefficient (PCC) between \hat{p}_{0, l}_{_{f}}_{, m} and α_{f, l}_{_{f}} |
| | (Eqn. 6) |
| [symbol] | The regularized PCC (PCCR) between \hat{p}_{0, l}_{_{f}}_{, m} and α_{f, l}_{_{f}} |
| λ_{f} | The regularization parameter in PCCR |

### C. Functional Signal Extraction Process

In one aspect, a hybrid method may include a process that extracts functional signals from a series of reconstructed functional images through regularized correlation. Given a set of reconstructed functional images \hat{p}_{0,l}_{_{f}},l_{f}=1,2, . . . , L_{f} in functional imaging, this process may extract functional signals through regularized correlation. To explain certain details of the process,

$${sum}_{j} {a}_{j} = {\sum }_{j = 1}^{J} {a}_{j} , {mean}_{j} {a}_{j} = \frac{{sum}_{j} {a}_{j}}{J} , and {norm}_{j} {a}_{j} = \sqrt{{\sum }_{j = 1}^{J} {a}_{j}^{2}}$$

for a series of numbers αⱼ, j=1,2, . . . , J are defined. It is assumed that the functional signal has a profile α_{f,l}_{_{f}}, l_{f}=1, 2, . . . , L_{f}, normalized to

$${\overline{α}}_{f , {l}_{f}} = ( {α}_{f , {l}_{f}} - {mean}_{{l}_{f}^{′}} {α}_{f , {l}_{f}^{′}} ) {( {norm}_{{l}_{{f}^{′}}^{′}} ( {α}_{f , {l}_{{f}^{′}}^{′}} - {mean}_{{l}_{f}^{′}} {α}_{f , {l}_{f}^{′}} ) )}^{- 1} .$$

The m-th voxel has a value of \hat{p}_{0,l}_{_{f,}}ₘ in the l_{f}-th image \hat{p}_{0,l}_{_{f}}. The functional amplitude can be quantified at the m-th voxel through the Pearson correlation coefficient (PCC) between \hat{p}_{0,l}_{_{f,}}ₘ and α_{f,l}_{_{f}};

$${PCC}_{{l}_{f}} ( {\hat{p}}_{0 , {l}_{f} , m} , {α}_{f , {l}_{f}} ) = \frac{{sum}_{{l}_{f}} ( {\overline{α}}_{f , {l}_{f}} ( {\hat{p}}_{0 , {l}_{f} , m} - {mean}_{{l}_{f}^{′}} {\hat{p}}_{0 , {l}_{f}^{′} , m} ) )}{{norm}_{{l}_{f}} ( {\hat{p}}_{0 , {l}_{f} , m} - {mean}_{{l}_{f}^{′}} {\hat{p}}_{0 , {l}_{f}^{′} , m} )} , \\ m = 1 , 2 , … , M , \qquad ( Eqn . 6 )$$

which is not robust for very-low-amplitude regions. To improve the robustness, a regularization term is added to the denominator and the regularized PCC (PCCR) is obtained:

$${{PCCR}_{{λ}_{f}}}_{{l}_{f}} ( {\hat{p}}_{0 , {l}_{f} , m} , {α}_{f , {l}_{f}} ) = \frac{{sum}_{{l}_{f}} ( {\overline{α}}_{f , {l}_{f}} ( {\hat{p}}_{0 , {l}_{f} , m} - {mean}_{{l}_{f}^{′}} {\hat{p}}_{0 , {l}_{f}^{′} , m} ) )}{\begin{matrix}{norm}_{{l}_{f}} ( {\hat{p}}_{0 , {l}_{f} , m} - {mean}_{{l}_{f}^{′}} {\hat{p}}_{0 , {l}_{f}^{′} , m} ) + \\ {λ}_{f} {mean}_{{m}^{′}} {norm}_{{l}_{f}} ( {\hat{p}}_{0 , {l}_{f} , {m}^{′}} - {mean}_{{l}_{f}^{′}} {\hat{p}}_{0 , {l}_{f}^{′} , {m}^{′}} )\end{matrix}} . m = 1 , 2 , … , M . \qquad ( Eqn . 7 )$$

A 3D functional image is formed by assigning the regularized correlation to each voxel.

The assumed functional signal profile α_{f,l}_{_{f}} is directly available in the numerical simulations provided by Eqn. S37. For mouse brain functional imaging in vivo, am, is let to be a sinusoidal profile synchronized with the paw stimulation pattern and it is applied to the UBP reconstructed images with 4N_{loc}=76 to obtain the functional region. The true functional profile is extracted from this region and it is applied to images with 4N_{loc}=40, 20, and 12.

### III. Supplemental Material

In Section III, examples of TISMC methods and hybrid methods are discussed.

### A. Exemplary Fast Forward Operator for a TISMC Method Based on Singular Value Decomposition (SVD) and Fast Fourier Transform (FFT)

In this Section III (A), an example of a fast forward operator for a TISMC method, according to various embodiments, that includes singular value decomposition (SVD) and fast Fourier transform (FFT) is discussed for application with the PACT system described in Lin, L. et al. “High-speed three-dimensional photoacoustic computed tomography for preclinical research and clinical translation,” Nat. Commun. Vol. 12, pp. 1-10 (2021). Similar fast forward operators can be determined for application with other tomographic imaging systems, shown below in Section IV.

In a homogeneous medium, a photoacoustic wave can be expressed as:

$$p ( r , t ) = \frac{1}{4 π {c}^{2}} {\int }_{V} \frac{{p}_{0} ( {r}^{′} )}{‖ {r}^{′} - r ‖} \frac{\partial }{\partial t} δ ( t - \frac{‖ {r}^{′} - r ‖}{c} ) {dr}^{′} . \qquad ( Eqn . S1 )$$

Here, p(r,t) is the pressure at location r and time t, c is the speed of sound (SOS), V is the volumetric space occupied by the tissue, and p₀(r′) is the initial pressure at r′. Assume the PACT system includes N finite-size ultrasonic transducer elements. For the n-th transducer element at rₙ with detection surface Sn, the average pressure on the detection surface at time t is expressed as

$$\tilde{p} ( {r}_{n} , t ) = \frac{1}{{A}_{n}} {\int }_{{S}_{n}} p ( r , t ) d {a}_{n} ( r ) . \qquad ( Eqn . S2 )$$

Here, Aₙ denotes the area of the surface Sp. The spatial impulse response (SIR) in Eqn. S2 is denoted as

Then Eqn. S2 Becomes:

$${h}_{s , n} ( {r}^{′} , τ ) = {\int }_{{S}_{n}} \frac{δ ( t - \frac{‖ {r}^{′} - r ‖}{c} )}{2 π c ‖ {r}^{′} - r ‖} d {a}_{n} ( r ) . \qquad ( Eqn . S3 )$$

$$\tilde{p} ( {r}_{n} , t ) = \frac{1}{2 c} {\int }_{V} {p}_{0} ( {r}^{′} ) \frac{1}{{A}_{n}} \frac{\partial }{\partial t} [ {\int }_{{s}_{n}} \frac{δ ( t - \frac{‖ {r}^{′} - r ‖}{c} )}{2 π c ‖ {r}^{′} - r ‖} d {a}_{n} ( r ) ] {dr}^{′} = \frac{1}{2 c} {\int }_{V} {p}_{0} ( {r}^{′} ) \frac{1}{{A}_{n}} \frac{\partial }{\partial t} {h}_{s , n} ( {r}^{′} , t ) {dr}^{′} . \qquad ( Eqn . S4 )$$

The electric impulse response (EIR) of the n-th transducer is denoted as h_{e,n}(t) and the transducer's response using temporal convolution*t is expressed as:

$$\hat{p} ( {r}_{n} , t ) = \tilde{p} ( {r}_{n} , t ) {*}_{t} {h}_{e , n} ( t ) . \qquad ( Eqn . S5 )$$

Substituting Eqn. S4 into Eqn. S5 yields:

$$\hat{p} ( {r}_{n} , t ) = \frac{1}{2 c} {\int }_{V} {p}_{0} ( {r}^{′} ) \frac{1}{{A}_{n}} \frac{\partial }{\partial t} {h}_{s , n} ( {r}^{′} , t ) {dr}^{′} {*}_{t} {h}_{e , n} ( t ) = {\int }_{V} {p}_{0} ( {r}^{′} ) \frac{{h}_{e , n}^{′} ( t ) {*}_{t} {h}_{s , n} ( {r}^{′} , t )}{2 c {A}_{n}} {dr}^{′} = {\int }_{V} {p}_{0} ( {r}^{′} ) {h}_{n} ( {r}^{′} , t ) {dr}^{′} . \qquad ( Eqn . S6 )$$

Here, the prime in h′_{e,n}(t) denotes the time derivative, and hₙ denotes the point source response per unit initial pressure per unit infinitesimal tissue volume received by a finite-size transducer element:

$${h}_{n} ( {r}^{′} , t ) = \frac{{h}_{e , n}^{′} ( t ) {*}_{t} {h}_{s , n} ( {r}^{′} , t )}{2 c {A}_{n}} . \qquad ( Eqn . S7 )$$

For convenience in the following discussion, t h_{s,n}(r′, t) and hₙ (r′, t) for r′ are temporally shifted such that time 0 aligns with the onset of the nonzero signal (arrival time) received by the center of the n-th transducer element rₙ:

$${\hat{h}}_{s , n} ( {r}^{′} , t ) = {h}_{s , n} ( {r}^{′} , t + \frac{‖ {r}^{′} - {r}_{n} ‖}{c} ) = {\int }_{{S}_{n}} \frac{δ ( t + \frac{‖ {r}^{′} - {r}_{n} ‖}{c} - \frac{‖ {r}^{′} - r ‖}{c} )}{2 π c ‖ {r}^{′} - r \vert \vert} {da}_{n} ( r ) \qquad ( Eqn . S8 )$$

$$and$$

$${\hat{h}}_{n} ( {r}^{′} , t ) = {h}_{n} ( {r}^{′} , t + \frac{‖ {r}^{′} - {r}_{n} ‖}{c} ) = \frac{{h}_{e , n}^{′} ( t ) {*}_{t} {h}_{s , n} ( {r}^{′} , t + \frac{‖ {r}^{′} - {r}_{n} ‖}{c} )}{2 c {A}_{n}} = \frac{{h}_{en}^{′} ( t ) {*}_{t} {\hat{h}}_{s , n} ( {r}^{′} , t )}{2 c {A}_{n}} . \qquad ( Eqn . S9 )$$

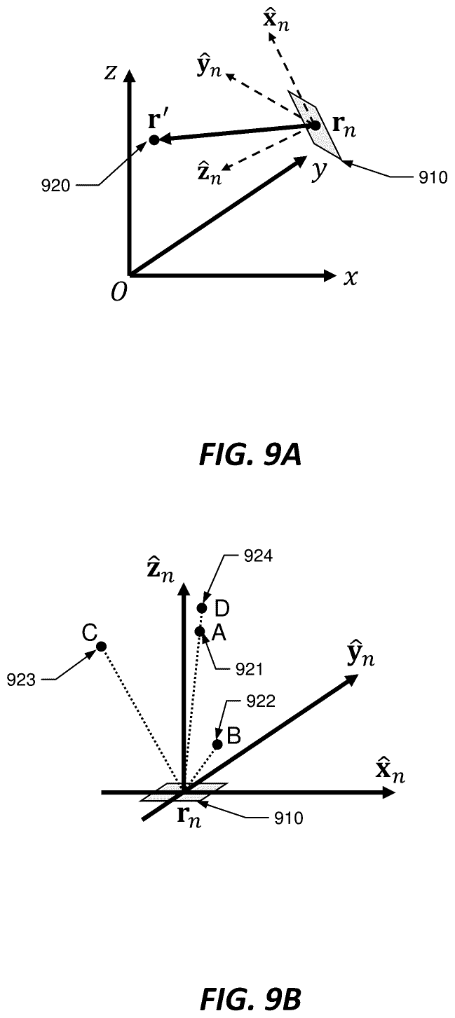

Next, the SIR and point source response are expressed in the local coordinates of the transducer elements. Each transducer element in this example has a flat rectangular detection surface with a length a of 0.7 mm and a width b of 0.6 mm, yielding Aₙ=ab=0.42 mm², n=1, 2, . . . , N. These transducer elements also have the same EIR: h_{e,n}(t)=hₑ(t), n=1, 2, . . . , N, and the measurement of hₑ(t) will be discussed in Section III (B). The local coordinates of the n-th transducer element are defined using the center the detection surface as the origin, the length direction as the x-axis, the width direction as the y-axis, and the normal direction (toward the detection region) as the z-axis. The x-axis and y-axis are selected to let the coordinates satisfy the right-hand rule. The three axes of the local coordinates are expressed as three vectors of unit length: \hat{x}ₙ, ŷₙ, and \hat{z}ₙ (shown in FIG. 9A), which form an orthonormal matrix:

$${A}_{n} = ( {\hat{x}}_{n}^{T} , {\hat{y}}_{n}^{T} , {\hat{z}}_{n}^{T} ) . \qquad ( Eqn . S10 )$$

Locations r′, rₙ, and r in the global coordinates correspond to the locations r′_{|c|}, 0, and r_{|c|} in the local coordinates of the n-th transducer element. Coordinate transformations yield r′_{|c|}=(r′−rₙ) Aₙ and r_{|c|}=(r−rₙ) Aₙ. These global and local coordinates satisfy ∥r′−rₙ∥=∥r′_{|c|}∥ and ∥r′−r∥=∥r′_{|c|}−r_{|c|} due to the orthonormality of the transformations. The detection surface (Sₙ in the global coordinates) is denoted in the local coordinates as S_{n,|c|}, and the local Leibniz notation is denoted as da_{n,|c|}(r_{|c|})=daₙ(r). Thus, in the local coordinates of the n-th transducer element, Eqn. S8 is expressed as:

$${\hat{h}}_{s , n} ( {r}^{′} , t ) = {\int }_{{S}_{n , lcl}} \frac{δ ( t + \frac{‖ {r}_{lcl}^{′} ‖}{c} - \frac{‖ {r}_{lcl}^{′} - {r}_{lcl} ‖}{c} )}{2 π c ‖ {r}_{lcl}^{′} - {r}_{lcl} ‖} {da}_{n , lcl} ( {r}_{lcl} ) , {r}_{lcl}^{′} = ( {r}^{′} - {r}_{n} ) {A}_{{r}_{n}} , n = 1 , 2 , … , N . \qquad ( Eqn . S11 )$$

All transducer elements are geometrically identical and have the same local coordinates, meaning S_{n,|c|}=S_{n′,|c|} and da_{n,|c|}(r_{|c|})=da_{n′,|c|}(r_{|c|}) for n, n′∈{1, 2, . . . , N}. Define S_{|c|}=S_{1,|c|}, da_{|c|}(r_{|c|})=da_{1,|c|}(r_{|c|}), and rewrite Eqn. S11 as:

$${\hat{h}}_{s , lcl} ( {r}_{lcl}^{′} , t ) = {\int }_{{S}_{lcl}} \frac{δ ( t + \frac{‖ {r}_{lcl}^{′} ‖}{c} - \frac{‖ {r}_{lcl}^{′} - {r}_{lcl} ‖}{c} )}{2 π c \vert \vert {r}_{lcl}^{′} - {r}_{lcl} ‖} {da}_{lcl} ( {r}_{lcl} ) , \qquad ( Eqn . S12 )$$

which is now independent of the transducer element index n. Replacing h′_{e,n}(t) and ĥ_{s,n}(r′, t) with h′ₑ(t) and ĥ_{s,|c|}(r′_{|c|}, t), respectively, in Eqn. S9, define:

$${\hat{h}}_{lcl} ( {r}_{lcl}^{′} , t ) = \frac{{h}_{e}^{′} ( t ) {*}_{t} {\hat{h}}_{s , lcl} ( {r}_{lcl}^{′} , t )}{2 c a b} . \qquad ( Eqn . S13 )$$

Thus, it is needed to calculate only the values of ĥ_{s,|c|}(r′_{|c|}, t) and ĥ_{|c|}(r′_{|c|}, t), then obtain the values of ĥ_{s,n}(r′, t) and ĥₙ (r′, t) through coordinate transformation:

$${\hat{h}}_{s , n} ( {r}^{′} , t ) = {\hat{h}}_{s , lcl} ( {r}_{lcl}^{′} , t ) \qquad ( Eqn . S 14 )$$

$$and$$

$${\hat{h}}_{n} ( {r}^{′} , t ) = \frac{{h}_{e , n}^{′} ( t ) {*}_{t} {\hat{h}}_{s , n} ( {r}^{′} , t )}{2 c {A}_{n}} = \frac{{h}_{e}^{′} ( t ) {*}_{t} {\hat{h}}_{s , lcl} ( {r}_{lcl}^{′} , t )}{2 c a b} = {\hat{h}}_{lcl} ( {r}_{lcl}^{′} , t ) , \qquad ( Eqn . S 15 )$$

respectively, with r′_{|c|}=(r′−rₙ) Aₙ for n=1, 2, . . . , N. Through these relations, both the SIR and the point source response are expressed in the local coordinates.

FIG. 9A depicts an illustration of an n-th transducer element (a black rectangular centered at rₙ) 910, a single point source 920, and n-th transducer element local coordinate system with axes \hat{x}ₙ, ŷₙ, and \hat{z}ₙ, according to an embodiment. FIG. 9B depicts an illustration of four point sources: a first point source 921, a second point source 922, a third point source 923, and a fourth point source 924 in the local coordinate system of the n-th transducer element 910, according to an embodiment. Points A, D, and rₙ are on the same line.

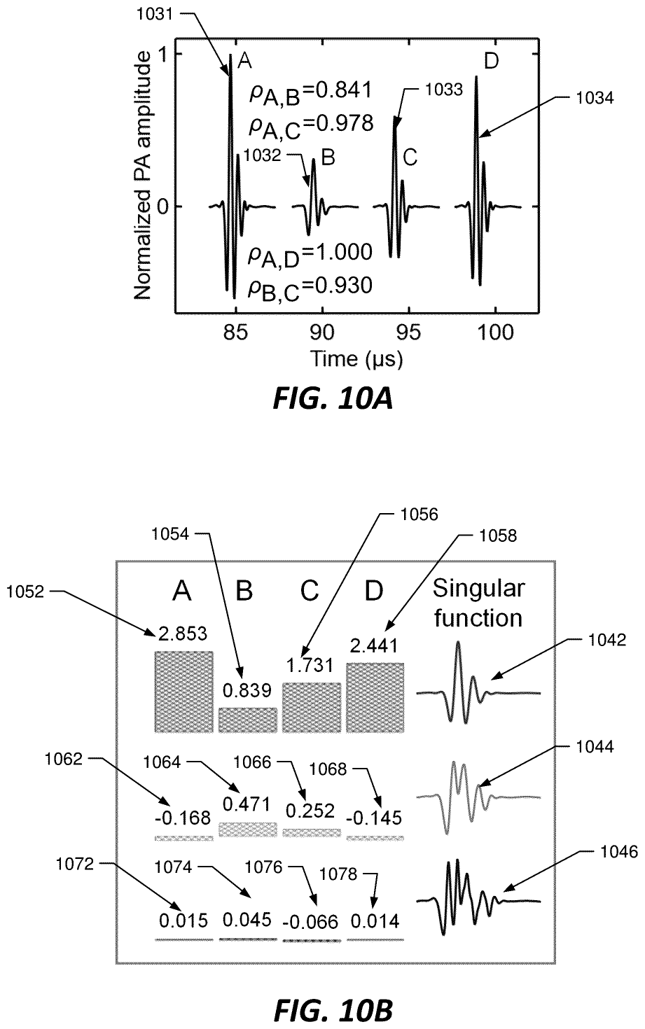

FIG. 10A depicts a graph of the responses 1031, 1032, 1033, and 1034 of the transducer element 910 in to the signals from the four point sources 921, 922, 923, and 924 in FIG. 9B and the PCC between every two responses. The time differences between responses are due to the differences in distances between voxel and transducer element. FIG. 10B depicts an illustration of four responses to the signals from the four point sources 921, 922, 923, and 924 in FIG. 9B using linear combinations (coefficients visualized with bars) of three temporal singular functions 1042, 1044, and 1046, respectively, based on a SVD procedure according to an embodiment. The illustration shows coefficients 1052, 1054, 1056, and 1058 for the first linear combination for the first singular function 1042, coefficients 1062, 1064, 1066, and 1068 for the second linear combination for the second singular function 1044, and coefficients 1072, 1074, 1076, and 1078 for the third linear combination for the third singular function 1046.

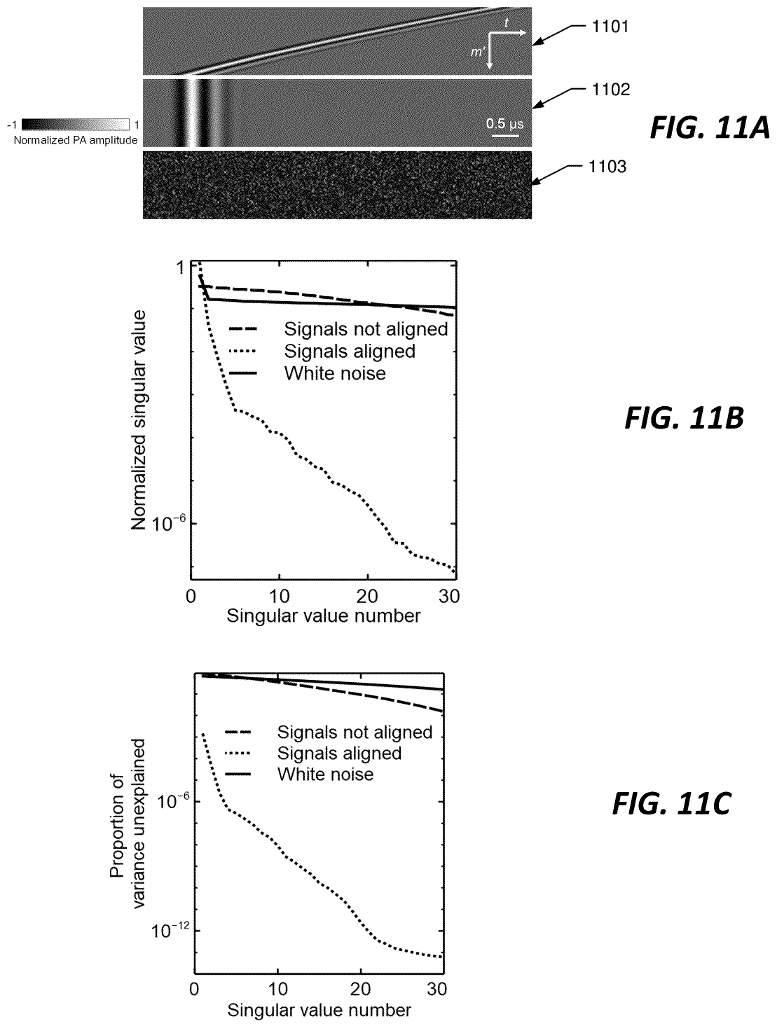

FIG. 11A depicts an illustration 1101 of response of a transducer element to fifty (50) point sources with decreasing distances to the element, an illustration 1102 of the temporally-shifted form which aligns the non-zero signals in time, and an illustration 1103 of white noise responses of the same size of as those in illustration 1102, according to an embodiment. FIG. 11B depicts a graph with normalized singular values in the SVDs of the signals in illustrations 1101, 1102, and 1103, according to an embodiment. FIG. 11C depicts a graph with proportions of the variances unexplained in the SVDs of the signals in illustrations 1101, 1102, and 1103, according to an embodiment.

In the illustrated example shown in FIG. 9B, the signals detected by a transducer element can be visualized by picking four point sources four point sources, labeled as A, B, C, and D, respectively, in the local coordinate system of the n-th transducer element, with points A, D, and the element center In on the same line. FIG. 10A depicts a plot of the element's responses to the signals from the point sources 921, 922, 923, and 924 in FIG. 9B. ρ_{A,B} denotes the Pearson correlation coefficient (PCC) between the responses corresponding to A and B. A direct implementation of the forward operator based on Eqn. S6 is computationally intensive. Although ρ_{A,D}=1, due to the effects of SIR, ρ_{A,B}, ρ_{A,C}, and ρ_{B,C} are less than 1, indicating the signals from points A, B, and C are not shift-invariant; thus, an efficient temporal convolution with one kernel function cannot yield the detected signals accurately.

To accelerate the forward operator, the spatial and temporal dimensions of ĥ_{|c|}(r′_{|c|},t) may be decoupled using singular value decomposition (SVD) while keeping only the dominant components:

$${\hat{h}}_{lcl} ( {r}_{lcl}^{′} , t ) \approx {\sum }_{k = 1}^{K} {\hat{h}}_{lcl , k} ( {r}_{lcl}^{′} ) {η}_{k} ( t ) . \qquad ( Eqn . S 16 )$$

Here, ĥ_{|c|,k}(r′_{|c|}) and ηₖ denote the k-th spatial singular function and the k-th temporal singular function, respectively; and the first K terms are used to approximate the whole series. Combining Eqns. S9, S15, and S16, the following can be obtained:

$${h}_{n} ( {r}^{′} , t ) = {\hat{h}}_{n} ( {r}^{′} , t - \frac{‖ {r}^{′} - {r}_{n} ‖}{c} ) = {\hat{h}}_{lcl} ( ( {r}^{′} - {r}_{n} ) {A}_{n} , t - \frac{‖ {r}^{′} - {r}_{n} ‖}{c} ) \approx {\sum }_{k = 1}^{K} {\hat{h}}_{lcl , k} ( ( {r}^{′} - {r}_{n} ) {A}_{n} ) {η}_{k} ( t - \frac{‖ {r}^{′} - {r}_{n} ‖}{c} ) . \qquad ( Eqn . S 17 )$$

Substituting Eqn. 17 into Eqn. S6, the following can be obtained:

$$\hat{p} ( {r}_{n} , t ) \approx {\int }_{V} {p}_{0} ( {r}^{′} ) {\sum }_{k = 1}^{K} {\hat{h}}_{lcl , k} ( ( {r}^{′} - {r}_{n} ) {A}_{n} ) {η}_{k} ( t - \frac{‖ {r}^{′} - {r}_{n} ‖}{c} ) {dr}^{′} = {\sum }_{k = 1}^{K} {η}_{k} ( t ) {*}_{t} {\int }_{V} {p}_{0} ( {r}^{′} ) {\hat{h}}_{lcl , k} ( ( {r}^{′} - {r}_{n} ) {A}_{n} ) δ ( t - \frac{‖ {r}^{′} - {r}_{n} ‖}{c} ) {dr}^{′} , n = 1 , 2 , … , N , t \geq 0. \qquad ( Eqn . S 18 )$$

As shown in this equation, the temporal variable is split from the spatial integrals, which allows for a fast implementation of the forward operator.

For an initial demonstration, Eqn. S16 with K=3 is applied to the responses 1031, 1032, 1033, and 1034 in FIG. 10A. FIG. 10B depicts an illustration of the three temporal singular functions η₁(t) 1042, η₂(t) 1044, η₃(t) 1046, respectively, and the values (coefficients) of the spatial singular functions 1042, 1044, 1046 are visualized as bars.

In various embodiments, a TISMC method includes temporal shifting for alignment. An example of temporal shifting for SVD is described in Eqns. S8 and S9. In one example, fifty (50) point sources were selected with decreasing distances to a transducer element and the element's independent responses to them were visualized as shown in illustration 1101 in FIG. 11A.

The temporally-shifted form of these responses based on Eqn. S9 is shown illustration 1102 in FIG. 11A. White-noise responses were added with the same size for comparison, as shown in illustration 1103 in FIG. 11A. Performing SVD to the three sets of responses, observed are different compression efficiencies from the perspectives of normalized singular value as shown in FIG. 11B and proportion of the variance in FIG. 11C. In both FIGS. 11B and 11C, it can be seen that the compression efficiency of the original responses is similar to that of the white-noise responses, whereas the efficiency for the temporally-shifted responses is significantly higher and thus advantageous for the compression.

B. Point Source Response of an Ultrasonic Transducer with a Flat Rectangular Detection Surface

In this Section III (B), an example of a point source (virtual transducer) system response for a virtual transducer element of an ultrasonic transducer with a flat rectangular detection surface is discussed.

The forward operator described in Section III (A) can be configured for an ultrasonic transducer with any detection surface. In the example discussed in this Section III (B), the ultrasonic transducer has a flat rectangular detection surface. From Eqns. S6 and S15 it is shown that the forward operator is based on the point source response ĝ_{|c|}(r′_{|c|}, t) in the local coordinates. The response is determined by the first derivative of EIR (h′ₑ(t)) and the realigned SIR (ĥ_{s,|c|}(r′_{|c|}, t)) in the local coordinates. In general, \ddot{h}_{|c|}(r′_{|c|}, t) can be measured experimentally. In this example, for an ultrasonic transducer with a flat rectangular detection surface, ĥ_{|c|}(r′_{|c|}, t) is calculated efficiently using the far-field approximation of ĥ_{s,|c|} (r′_{|c|}, t). From a special case of this approximation, a method is derived to measure h′ₑ(t) experimentally.

In the first step of obtaining ĥ_{|c|}(r′_{|c|}, t), the calculation of ĥ_{|c|}(r′_{|c|}, t) is introduced based on the far-field approximation of ĥ_{s,|c|} (r′_{|c|}, t). In this research, the image domain is within the far-field regions of all transducer elements. Using the far-field approximation, the SIR is expressed in the frequency domain as:

$${F}_{t} ( {h}_{s , n} ( {r}^{′} , t ) ) ( f ) \approx \frac{a b exp ( - j 2 π f \frac{‖ {r}^{′} - {r}_{n} ‖}{c} )}{2 π c ‖ {r}^{′} - {r}_{n} ‖} sin c ( π f \frac{a \vert ( {r}^{′} - {r}_{n} ) {\hat{x}}_{n}^{T} \vert}{c ‖ {r}^{′} - {r}_{n} ‖} ) sin c ( π f \frac{b \vert ( {r}^{′} - {r}_{n} ) {\hat{y}}_{n}^{T} \vert}{c ‖ {r}^{′} - {r}_{n} ‖} ) . \qquad ( Eqn . S 19 )$$

Here, Ft denotes the temporal FT, and j denotes the imaginary unit. Combining Eqns. S14 and S19, the temporal Fourier transform (FT) is applied to ĥ_{s,|c|}(r′_{|c|}, t) to obtain:

$${F}_{t} ( {\hat{h}}_{s , lcl} ( {r}_{lcl}^{′} , t ) ) ( f ) = {F}_{t} ( {h}_{s , n} ( {r}^{′} , t + \frac{‖ {r}^{′} - {r}_{n} ‖}{c} ) ) ( f ) = exp ( j 2 π f \frac{‖ {r}^{′} - {r}_{n} ‖}{c} ) {F}_{t} ( {h}_{s , n} ( {r}^{′} , t ) ) ( f ) \approx \frac{a b}{2 π c ‖ {r}^{′} - {r}_{n} ‖} sin c ( π f \frac{a \vert ( {r}^{′} - {r}_{n} ) {\hat{x}}_{n}^{T} \vert}{c ‖ {r}^{′} - {r}_{n} ‖} ) sin c ( π f \frac{b \vert ( {r}^{′} - {r}_{n} ) {\hat{y}}_{n}^{T} \vert}{c ‖ {r}^{′} - {r}_{n} ‖} ) = \frac{a b}{2 π c ‖ {r}_{lcl}^{′} ‖} sin c ( π f \frac{a \vert {x}_{lcl}^{′} \vert}{c ‖ {r}_{lcl}^{′} ‖} ) sin c ( π f \frac{b \vert {y}_{lcl}^{′} \vert}{c ‖ {r}_{lcl}^{′} ‖} ) . \qquad ( Eqn . S 20 )$$

Here, identities r′_{|c|}=(r′−rₙ) Aₙ=(r′−rₙ) (\hat{x}ₙ^{T}, ŷₙ^{T}, \hat{z}ₙ^{T})=(x′_{|c|}, y′_{|c|}, z′_{|c|}) and ∥r\hat{ }′−r_n∥=∥r_|c|\hat{ }′∥, and r′_{|c|} must not be the origin in the local coordinates. Substituting Eqn. S20 into the temporal FT of Eqn. S13, obtains:

$${F}_{t} ( {\hat{h}}_{lcl} ( {r}_{lcl}^{′} , t ) ) ( f ) = \frac{1}{2 c a b} {F}_{t} ( {h}_{e}^{′} ( t ) ) ( f ) {F}_{t} ( {\hat{h}}_{s , lcl} ( {r}_{lcl}^{′} , t ) ) ( f ) \approx \frac{{F}_{t} ( {h}_{e}^{′} ( t ) ) ( f )}{4 π {c}^{2} ‖ {r}_{lcl}^{′} ‖} sin c ( π f \frac{a \vert {x}_{lcl}^{′} \vert}{c ‖ {r}_{lcl}^{′} ‖} ) sin c ( π f \frac{b \vert {y}_{lcl}^{′} \vert}{c ‖ {r}_{lcl}^{′} ‖} ) . \qquad ( Eqn . S 21 )$$

Applying temporal inverse FT to Eqn. S21, obtains:

$${\hat{h}}_{lcl} ( {r}_{lcl}^{′} , t ) \approx {F}_{t}^{- 1} ( \frac{{F}_{t} ( {h}_{e}^{′} ( t ) ) ( f )}{4 π {c}^{2} ‖ {r}_{lcl}^{′} ‖} sin c ( π f \frac{a \vert {x}_{lcl}^{′} \vert}{c ‖ {r}_{lcl}^{′} ‖} ) sin c ( π f \frac{b \vert {y}_{lcl}^{′} \vert}{c ‖ {r}_{lcl}^{′} ‖} ) ) . \qquad ( Eqn . S 22 )$$

In the second step of obtaining ĥ_{|c|}(r′_{|c|}, t), a method is derived to measure h′ₑ(t) by analyzing a special case of Eqn. S22. The location r′_{|c|} is constrained on the axis of the transducer by letting r′_{|c|}=(0,0, z′_{|c|}), which simplifies Eqn. S22 to:

$${\hat{h}}_{lcl} ( {r}_{lcl}^{′} , t ) \approx {F}_{t}^{- 1} ( \frac{{F}_{t} ( {h}_{e}^{′} ( t ) ) ( f )}{4 π {c}^{2} \vert {z}_{lcl}^{′} \vert} ) = \frac{{h}_{e}^{′} ( t )}{4 π {c}^{2} \vert {z}_{lcl}^{′} \vert} . \qquad ( Eqn . S 23 )$$

Solving for h′ₑ(t) from Eqn. S23, obtains:

$${h}_{e}^{′} ( t ) \approx 4 π {c}^{2} \vert {z}_{lcl}^{′} \vert {\hat{h}}_{lcl} ( {r}_{lcl}^{′} , t ) = 4 π {c}^{2} \vert {z}_{lcl}^{′} \vert {\hat{h}}_{n} ( {r}^{′} , t ) = 4 π {c}^{2} \vert {z}_{lcl}^{′} \vert {\hat{h}}_{n} ( ( 0 , 0 , {z}_{lcl}^{′} ) {A}_{{r}_{n}}^{- 1} + {r}_{n} , t + \frac{\vert {z}_{lcl}^{′} \vert}{c} ) . \qquad ( Eqn . S 24 )$$

Here, the identity r′=r′_{|c|}Aₙ⁻¹+rₙ=(0,0, z′_{|c|})Aₙ⁻¹+rₙ is used. In this example, the measurement of the right-hand side of Eqn. S24 is repeated and the average used to represent h′ₑ(t).

In summary, h′ₑ(t) is measured experimentally on the basis of Eqn. S24 and the measurement is substituted into Eqn. S22 to obtain ĥ_{|c|}(r′_{|c|}, t). Further, SVD is performed to ĥ_{|c|}(r′_{|c|}, t) according to Eqn. S16 and obtain singular functions ĥ_{|c|,k}(r′_{|c|}) and ηₖ(t), k=1,2, . . . , K are obtained. These functions in Eqn. S18 are used to implement the fast forward operator.

### C. Discretization of the Forward Operator

In this subsection III (C), a fast forward operator for a TISMC method of an embodiment is discretized for use to determine values that can be used, for example, to determine coefficients used in the forward simulation.

In certain examples, a forward operator of a TISMC method may be expressed in two different forms as provided in Eqns. S6 and S18, respectively. First, the forward operator implemented in Eqn. S6 is discretized. In the temporal domain, points of interest tₗ=t₁+(l−1)τ, l=1, 2, . . . , L, are chosen where t₁ is the initial time, τ is the sampling step size, and L is the number of time points. Then, the temporal FT of h′ₑ(t) as Fₜ(h′ₑ(t))_{l′}≈Fₗ(h′ₗ) (l′), l′=1,2, . . . , L, is discretized where h′ₗ=h′ₑ(tₗ), Fₜ(h′ₑ(t))_{l′}, =Fₜ(h′ₑ(t))(f_{l′}), l, l′∈{1, 2, . . . , L}, and Fₗ represents the discrete FT with respect to l. The temporal frequencies f_{l′}, l′=1,2, . . . , L are selected according to the requirement of the discrete FT. Further, the m_{|c|}-th location in the local coordinates is denoted as r_{|c|m}_{_{|c|}}=(x′_{|c|m}_{_{|c|}}, y′_{|c|m}_{_{|c|}}, z′_{|c|m}_{_{|c|}}) and ĥ_{|c|m}_{_{|c|}}=ĥ_{|c|}(r′_{|c|m}_{_{|c|}}, tₗ)m_{|c|}=1, 2, . . . , M_{|c|}, is defined where M_{|c|} is the number of locations of interest in the local coordinates. Thus, Eqn. S22 is discretized as:

$${\hat{h}}_{lcl , {m}_{lcl} , l} \approx \frac{1}{4 π {c}^{2} ‖ {r}_{lcl , {m}_{lcl}}^{′} ‖} {F}_{l}^{- 1} ( {F}_{l} ( {h}_{l}^{′} ) ( {l}^{′} ) sin c ( π {f}_{{l}^{′}} \frac{a \vert {x}_{lcl , {m}_{lcl}}^{′} \vert}{‖ {r}_{lcl , {m}_{lcl}}^{′} ‖} ) sin c ( π {f}_{{l}^{′}} \frac{b \vert {y}_{lcl , {m}_{lcl}}^{′} \vert}{c ‖ {r}_{lcl , {m}_{lcl}}^{′} ‖} ) ) ( l ) , {m}_{lcl} = 1 , 2 , … , {M}_{lcl} , l = 1 , 2 , … , L . \qquad ( Eqn . S 25 )$$

Further, the point source response hₙ (r′, t) are discretized as h_{n,m,l}=hₙ (r′ₘ, tₗ), n=1, 2, . . . , N, m=1, 2, . . . , M, l=1, 2, . . . , L. Here, M is the number of voxels (source points) in the image domain. On the basis of the relation

$${h}_{n} ( {r}_{m}^{′} , {t}_{l} ) = {\hat{h}}_{n} ( {r}_{m}^{′} , {t}_{l} - \frac{‖ {r}_{m}^{′} - {r}_{n} ‖}{c} ) = {\hat{h}}_{lcl} ( ( {r}_{m}^{′} - {r}_{n} ) {A}_{n} , {t}_{l} - \frac{‖ {r}_{m}^{′} - {r}_{n} ‖}{c} ) ,$$

the values of h_{n,m,l} are obtained through spatiotemporal interpolation of the values of ĥ_{|c|m}_{_{|c|}}_{,l}. Denoting \hat{p}_{n,l}=\hat{p}(rₙ, tₗ) and p_{0,m}=p_{0,m}(r′ₘ), Eqn. S6 is discretized as:

$${\hat{p}}_{n , l}^{slow} = {\sum }_{m = 1}^{M} {v}_{m} {h}_{n , m , l} {p}_{0 , m} , n = 1 , 2 , … , N , l = 1 , 2 , … , L . \qquad ( Eqn . S 26 )$$

Here, \hat{p}_{n,l}^{slow} represents an approximation of \hat{p}_{n,l} using this relatively slow operator, and vₘ represents the volume of the m-th voxel. In this example, due to the finite duration of every point source response shown in FIGS. 10A, in illustration 1101 of FIG. 11A, and in illustration 1102 of FIG. 11A, given a combination of m and n, it is needed to calculate only for l in a range of length L′<L. Here, L′ is the effective length for nonzero values in the discretized point source responses, and the computational complexity of the discrete forward operator in Eqn. S26 is O(NML′).

Next, the forward operator is discretized to a form with lower computational complexity from Eqn. S18. h̅_{|c|,k,m}_{_{|c|}}=ĥ_{|c|,k} (r′_{|c|,m}_{_{|c|}}) and η_{k,l}=ηₖ(tₗ), k=1, 2, . . . , K, m_{|c|}=1,2, . . . , M_{|c|}, l=1, 2, . . . , L. After obtaining the array ĥ_{|c|,m}_{_{|c|,}}ₗ through Eqn. S25, ĥ_{|c|,k,m}_{_{|c|}} and η_{k,l} are estimated through SVD:

$${\hat{h}}_{lcl , {m}_{lcl} , l} \approx {\sum }_{k = 1}^{K} {\overline{h}}_{lcl , k , {m}_{lcl}} {η}_{k , l} , {m}_{lcl} = 1 , 2 , … , {M}_{lcl} , l = 1 , 2 , … , L . \qquad ( Eqn . S 27 )$$

ĥ_{|c|,k,n,m}=ĥ_{|c|,k}((r′ₘ−rₙ)Aₙ), which is obtained from the values of h̅_{|c|,k,m}_{_{|c|}} through spatial interpolation. Moreover,

$$δ ( t - \frac{‖ {r}^{′} - {r}_{n} ‖}{c} )$$

is expressed in the discrete form as:

$$\frac{1}{τ} ( ( {t}_{{l}_{n , m} + 1} - \frac{‖ {r}_{m}^{′} - {r}_{n} ‖}{c} ) {δ}_{l , {l}_{n , m}} + ( \frac{‖ {r}_{m}^{′} - {r}_{n} ‖}{c} - {t}_{{l}_{n , m}} ) {δ}_{l , {l}_{n , m} + 1} ) , \\ n = 1 , 2 , … , N , m = 1 , 2 , … , M , l = 1 , 2 , … , L . \qquad ( Eqn . S28 )$$

Here, l_{n,m} denotes the temporal index such that

$${t}_{{l}_{n , m}} \leq \frac{‖ {r}_{m}^{′} - {r}_{n} ‖}{c} < {t}_{{l}_{n , m} + 1} ,$$

and the Kronecker delta function is used:

$${δ}_{l , {l}^{′}} = \{ \begin{matrix}0 , & l \neq {l}^{′} , \\ 1 , & l = {l}^{′}\end{matrix} . \qquad ( Eqn . S29 )$$

The forward operator in Eqn. S18 is discretized as:

$${\hat{p}}_{n , l}^{fast} = \qquad ( Eqn . S30 )$$

$${\sum }_{k = 1}^{K} {η}_{k , l} {*}_{l} {\sum }_{m = 1}^{M} {v}_{m} {\hat{h}}_{lcl , k , n , m} {p}_{0 , m} \frac{1}{τ} [ \begin{matrix}( {t}_{{l}_{n , m} + 1} - \frac{‖ {r}_{m}^{′} - {r}_{n} ‖}{c} ) {δ}_{l , {l}_{n , m}} + \\ ( \frac{‖ {r}_{m}^{′} - {r}_{n} ‖}{c} - {t}_{{l}_{n , m}} ) {δ}_{l , {l}_{n , m} + 1}\end{matrix} ] ,$$

$$n = 1 , 2 , … , N , l = 1 , 2 , … , L .$$

Here, \hat{p}_{n,l}^{fast} represents an approximation of \hat{p}_{n,l} using the fast operator, and *l denotes the discrete convolution with respect to l. In this example, a value of L is chosen so that log₂L is an integer, and the discrete convolution is implemented using the temporal fast Fourier transform (FFT). Thus, the computational complexity of the discrete forward operator in Eqn. S30 is O(NMK)+O(N (Llog₂L)K).

### D. Efficiency and Signal-Domain Accuracy of the Fast Forward Operator

In this Section III (D), the efficiency and signal-domain accuracy of a fast forward operator for an example of a TISMC method according to an embodiment is discussed.

To quantify the efficiency and accuracy of a fast forward operator for the TISMC method determined based on Eqn. S30, numerical simulations were performed by placing a numerical phantom of size 1×1×1 cm³ in four image subdomains D₁, D₂, D₃, and D₄ of a PACT system, according to an embodiment. FIG. 12A depicts an illustration of the numerical phantom formed by three rectangular cuboids (each of size 2.6×2.6×10 mm³) intersecting at their centers, according to an implementation. Voxel values are 1 inside the phantom and 0 outside. Three lines (L1, L2, and L3) are picked for image-domain comparisons shown in FIG. 14. FIG. 12B depicts a virtual 2D array (solid arcs), four subdomains D₁, D₂, D₃, and D₄, each of size 1×1×1 cm³, and the domain occupied by rotations of the four subdomains around the array axis (dotted circles and arcs), according to an implementation.

The virtual 2D array is formed by the rotation of the four arc arrays is marked by arcs and the rotation of the four subdomains around the same axis covers a domain (marked by dotted circles and arcs) enclosing all the image domains. A slow operator is implemented according to Eqn. S26 for comparison.

FIG. 12C depicts a plot of computation times (t_{fast} and t_{slow}) of the fast and slow operators, respectively, for the forward simulations with the numerical phantom in D₁ (36 repetitions), according to an implementation. FIG. 13A depicts images of relative errors of the simulated signals for all virtual elements (n′_{loc}=1,2, . . . , 396, n′ₑₗₑ=1,2, . . . , 128), with the numerical phantom in D₁, D₂, D₃, and D₄, according to an implementation. FIG. 13B depicts a comparison of the signals (\hat{p}^{slow} and \hat{p}^{fast}) simulated by the fast and slow operators for the 64-th element (n′ₑₗₑ=64) in the first virtual arc array (n′_{loc}=1), with the phantom in D₁, D₂, D₃, and D₄, respectively, in FIG. 13A.

The efficiency of the fast operator is quantified by performing forward simulations with the numerical phantom in the subdomain D₁ using both operators with parameters M=50×50×50 (voxel size of 0.2×0.2×0.2 mm³), N=396×128 (the four-arc array rotated for 99 locations and each arc downsized to 128 elements), L=4096, L′=151, and K=3. The single-thread-CPU version of each operator was run 36 times on a server with Ubuntu 20.04.6 LTS and Intel® Xeon® Gold 6248R CPU @ 3.00 GHz. Denoting the computation times of both operators as t_{fast} and t_{slow}, respectively, mean(t_{fast})=6.3 min, std(t_{fast})=0.4 min, mean (t_{slow})=267 min, and std (t_{slow})=19 min was obtained for the 36 simulations. A comparison of these values is shown in FIG. 12C. A Welch's t-test was performed between 42t fast and t_{slow}, showing insignificant difference (p-value=0.59). Thus, the fast operator with K=3 has approximately 42 times the speed of the slow operator, and the acceleration ratio can be further improved by reducing K.

Then, the accuracy of the fast operator is demonstrated by performing forward simulations for the numerical phantom at the four subdomains (all the other parameters are the same as above). The results from the slow operator are regarded as ground truth (\hat{p}_{gt,n,l}=\hat{p}_{n,l}^{slow}) and the relative error of the fast-operator results (\hat{p}_{n,l}^{fast}) is defined as

$${e}_{n} = \frac{\sqrt{{\sum }_{l = 1}^{L} {( {\hat{p}}_{n , l}^{fast} - {\hat{p}}_{gt , n , l} )}^{2}}}{\sqrt{{\sum }_{l = 1}^{L} {\hat{p}}_{gt , n , l}^{2}}} , n = 1 , 2 , … , N , l = 1 , 2 , … , L .$$

The values of relative error eₙ for the four subdomains (D₁, D₂, D₃, and D₄) are compared in FIG. 14, with n′_{loc} the index of the virtual arc array and n′ₑₗₑ the index of the element in each array, n′_{loc}=1,2, . . . , 396, n′ₑₗₑ=1,2, . . . , 128. These relative errors are generally small and all of them are below 0.005, which is enough for this study. For the 64-th element (n′ₑₗₑ=64) on the first virtual array (n′_{loc}=1), the signals generated by the slow and fast operators were compared, denoted as \hat{p}^{slow} and \hat{p}^{fast} respectively, which show the fast operator's high accuracy across the time domain.

FIG. 14 are plots of the relative error of the reconstructed image in subdomains D₁, D₂, D₃, and D₄ with 1 to 256 iterations, values on the lines L1, L2, and L3 in the reconstructed images with ηᵢₜₑᵣ=1, 4, 8, 64, 256, and a comparison between ground truth (p_{0,L1}, p_{0,L2}, and p_{0,L3}) and 256-iteration reconstructed (\hat{p}_{0,L1}, \hat{p}_{0,L2}, and \hat{p}_{0,L3}) values along the three lines, according to an implementation.

### E. Regularized Iterative Reconstruction and Image-Domain Accuracy of the Fast Forward Operator of TISMC Methods

In this Section III (E), the image-domain accuracy of a fast forward operator for an example of a TISMC method according to an embodiment is discussed.

The discretized initial pressure and transducer response is denoted as column vectors p₀ and \hat{p}, respectively, and the forward model is expressed as:

$$\hat{p} = {Hp}_{0} , \qquad ( Eqn . S31 )$$

where H is the discretized system matrix of shape (NL, M). If H is full column rank (columns of the matrix are linearly independent), an image may be reconstructed by solving the optimization problem:

$${\hat{p}}_{0} = {arg min}_{{p}_{0} ∈ {ℝ}^{M}} {‖ {Hp}_{0} - \hat{p} ‖}^{2} \qquad ( Eqn . S32 )$$

using FISTA with a constant step size, according to an embodiment.

The Lipschitz constant (the largest eigenvalue) of 2H^{T}H may be obtained through the power method with, e.g., 32 iterations and it may be used in FISTA. Further, nonnegativity of the initial pressure may be added as a constraint to facilitate the reconstruction:

$${\hat{p}}_{0} = {arg min}_{{p}_{0} ∈ {ℝ}^{M} , {p}_{0} \geq 0} {‖ {Hp}_{0} - \hat{p} ‖}^{2} . \qquad ( Eqn . S33 )$$

If H is not full column rank or the noise level is high, the image may be reconstructed by solving the regularized optimization problem:

$${\hat{p}}_{0} = {arg min}_{{p}_{0} ∈ {ℝ}^{M} , {p}_{0} \geq 0} {γ}_{c} {‖ {Hp}_{0} - \hat{p} ‖}^{2} + λ {\vert {p}_{0} \vert}_{TV} \qquad ( Eqn . S34 )$$

using FISTA with a constant step size. Here, λ denotes the regularization parameter and γ_{c} represents the system-specific measurement calibration factor. In one example, instead of dealing with \hat{p} directly, γ_{c}\hat{P}₀ from γ_{c}\hat{P} (reading of the data acquisition system) are obtained by solving the problem

$${γ}_{c} {\hat{p}}_{0} = {arg min}_{{γ}_{c} {p}_{0} ∈ {ℝ}^{M} , {γ}_{c} {p}_{0} \geq 0} {‖ H ( {γ}_{c} {p}_{0} ) - {γ}_{c} \hat{p} ‖}^{2} + λ {\vert {γ}_{c} {p}_{0} \vert}_{TV} ,$$

which is equivalent to Eqn. S34. The normalized values of γ_{c}\hat{P}₀ (the same as those of \hat{P}₀) are used for further analysis, and effect of γ_{c} is ignored in this example. The term |p₀|_{TV} in Eqn. S34 denotes the total variation (TV) norm, defined as:

$${\vert {p}_{0} \vert}_{TV} = {\sum }_{\begin{matrix}2 \leq {m}_{1} \leq {M}_{1} \\ 2 \leq {m}_{2} \leq {M}_{2} \\ 2 \leq {m}_{3} \leq {M}_{3}\end{matrix}} \sqrt{\begin{matrix}{( \frac{{p}_{0 , {m}_{1} , {m}_{2} , {m}_{3}} - {p}_{0 , {m}_{1} - 1 , {m}_{2} , {m}_{3}}}{{v}_{m , 1}} )}^{2} + \\ {( \frac{{p}_{0 , {m}_{1} , {m}_{2} , {m}_{3}} - {p}_{0 , {m}_{1} , {m}_{2} - 1 , {m}_{3}}}{{v}_{m , 2}} )}^{2} + \\ {( \frac{{p}_{0 , {m}_{1} , {m}_{2} , {m}_{3}} - {p}_{0 , {m}_{1} , {m}_{2} , {m}_{3} - 1}}{{v}_{m , 3}} )}^{2}\end{matrix}} . \qquad ( Eqn . S35 )$$

In this definition, v_{m,1}, v_{m,2}, and v_{m,3} represent the voxel sizes along the x-axis, y-axis, and z-axis, respectively; M₁, M₂, and M₃ (M=M₁M₂M₃) denote the number of voxels in the first, second, and third dimensions, respectively; and the column vector p₀ to a 3D array p_{0,M}_{₁}_{×M}_{₂}_{×M}_{₃} is reshaped to express 3D information. Given a set of parameters for FISTA, the solution to Eqn. S32 is linearly dependent on p. The solutions to Eqns. S33 and S34 are nonlinearly dependent on p due to the nonnegative constraint (p₀≥0) and the TV regularization. Symbols for the forward operator and image reconstruction are summarized in Table 1.

The accuracy of the fast forward operator may be quantified in the image domain by performing forward simulations using the slow operator (H implemented through Eqn. S26, regarded as ground truth) and image reconstructions using iterations with nonnegativity constraints (Eqn. S33 with H implemented through Eqn. S30 and accelerated by two NVIDIA A 100 GPUS), with the numerical phantom shown in FIG. 12A in the four subdomains D₁, D₂, D₃, and D₄, respectively. The reconstructed image \hat{p}₀'s relative error to the ground-truth image p_{0,gt} is defined as

$$\frac{‖ {\hat{p}}_{0} - {p}_{0 , gt} ‖}{‖ {p}_{0 , gt} ‖} .$$

For the reconstructions with the numerical phantom in D₁, the relative error reduces to 0.003 as the iteration number ηᵢₜₑᵣ reaches 256, as shown in FIG. 14. In FIG. 14, the values of the reconstructed images are plotted along three lines (L1, L2, and L3) for ηᵢₜₑᵣ=1, 4, 8, 64, 256. Further, the ground-truth (256-iteration reconstructed) values along the lines are denoted as p_{0,L1}, p_{0,L2}, and p_{0,L3} (\hat{p}_{0,L1}, \hat{p}_{0,L2}, and \hat{p}_{0,L3}), respectively, and compared in FIG. 14. All the plots show the high accuracy of the fast forward operator in the image domain.
F. Iterative Reconstruction with Noisy Data or Sparse Sampling

In this Section III (F), the fast forward operator for an example of a TISMC method according to an embodiment is discussed under the condition of noisy data and sparse sampling. Regularization is used in the iterative method to stabilize the image reconstruction.

After validating the accuracy of the fast forward operator in both the signal domain and image domain in Section III (E), its GPU-accelerated implementation was used for both forward simulation and image reconstruction in the following analyses.

First, a forward simulation is performed with the numerical phantom (e.g., numerical phantom shown in FIG. 12A) in the subdomain D₁ (e.g., subdomain D₁ shown in FIG. 12B), white noise is added with different amplitudes to the signals, and the images reconstructed through iterations with nonnegativity constraint (Eqn. S33) and iterations with TV regularization (Eqn. S34), respectively. The amplitude of the signal is defined as half of the difference between its maximum and minimum values, the amplitude of noise is defined as its standard deviation, and their division is defined as signal-to-noise ratio (SNR). The relative error

$$\frac{‖ {\hat{p}}_{0} - {p}_{0 , gt} ‖}{‖ {p}_{0 , gt} ‖}$$

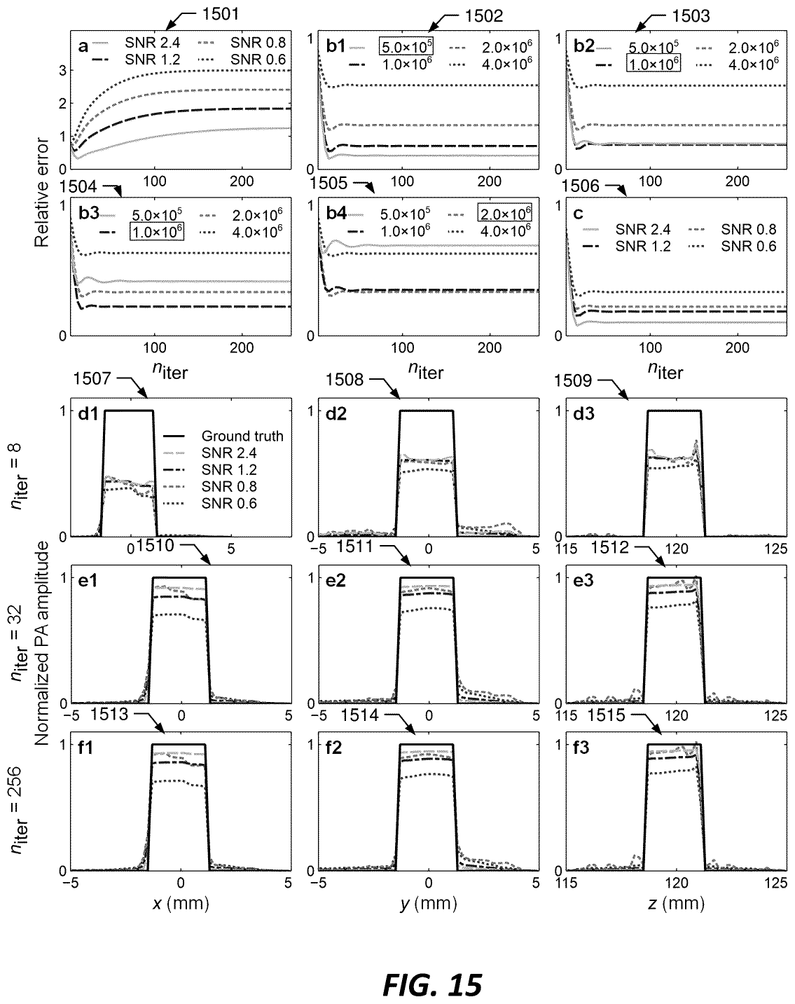

between the reconstructed image \hat{p}₀ and ground truth p_{0,gt} is used to quantify the accuracy. For SNRs of 2.4, 1.2, 0.8, and 0.6, the relative errors of the images reconstructed without TV regularization are shown in plot 1501, which means that the reconstructions are unsuccessful. For iterations with TV regularization, the relative errors for regularization parameters of 5.0×10⁵, 1.0×10⁶, 2.0×10⁶, and 4.0×10⁶ (mm) are shown in plots 1502, 1503, 1504, and 1505 for the four SNRs, respectively, which demonstrate that TV regularization stabilizes the iterations and the best choice of regularization parameter (enclosed in black-solid boxes) is noise level dependent. Plots with the best regularization parameters in plots 1502, 1503, 1504, and 1505 are summarized in a plot 1506, which satisfies that the higher the signal noise level the poorer the image quality. Values on the lines L1, L2, and L3 in FIG. 12B in the reconstructed images after 8, 32, and 256 iterations are shown in plots 1507, 1508, 1509, 1510, 1511, 1512, 1513, 1514, and 1515 in FIG. 15, respectively. The fact that 8 iterations already reconstruct a large portion of the image inspires us to perform only 8 iterations in certain steps of the motion correction discussed below, where the general structure is more important than accurate amplitude. The fact that 256 iterations bring minor improvements to 32 iterations inspires us to perform only 32 iterations for image reconstruction in functional imaging and the final step of motion correction as discussed below.

FIG. 15 includes plots of results of iterative reconstruction for noisy data, according to an implementation. FIG. 15 includes a plot 1501 of relative errors of the images of the numerical phantom in D₁ reconstructed iteratively with nonnegativity constraint, for SNRs of 2.4, 1.2, 0.8, and 0.6. FIG. 15 includes plots 1502, 1503, 1504, and 1505 of relative errors of the images reconstructed iteratively with TV regularization, for SNRs of 2.4, 1.2, 0.8, and 0.6, respectively, for different choices of regularization parameters (mm). FIG. 15 includes a plot 1506 of a summary of the plots in plots 1502, 1503, 1504, and 1505 with the best choices of regularization parameters (enclosed in black-solid boxes). Plots 1507, 1508, 1509, 1510, 1511, 1512, 1513, 1514, and 1515 include values on the lines L1, L2, and L3 in the images reconstructed with 8, 32, and 256, iterations, respectively.

Then a regularized iterative method was used to reconstruct a mouse brain numerical phantom with signals from the phantom detected by virtual arrays with different numbers of arcs: 4N_{loc}=76, 40, 28, 20, 16, and 12, as shown in the fourth column in FIG. 5. Maximum amplitude projections (MAPs) of the images (along the z-axis) reconstructed by regularized iterative method with different regularization parameters for 4N_{loc}=76, 40, 28, 20, 16, and 12 are shown in FIG. 16. Here, instead of selecting the regularization parameters quantitatively according to the relative error as in plots 1502, 1503, 1504, and 1505 in FIG. 15, they are selected qualitatively by balancing mitigating artifacts and maintaining image resolution, which mimics the reality where the ground truth is unknown. The best images are indicated by red-solid boxes in FIG. 16.

FIG. 16 depicts an illustration including regularized iterative reconstruction of mouse brain images with sparse sampling, according to an implementation. FIG. 17 depicts a flowchart of a hybrid method for image reconstruction from sparsely sampled raw data and a prior image, according to an embodiment. FIG. 18 depicts MAPs of the images shown in FIG. 5.

FIGS. 19A-C depict illustrations of the results of a linearity test of UBP and regularized iterative method, according to an embodiment. FIG. 19A depicts images of the first and second numerical phantoms and their sum, respectively, and an image domain (a rectangular cuboid with red-solid edges) and two virtual arrays: all arcs (4M_{loc}=76) and the arcs with red boundaries (4N_{loc}=12), according to an embodiment. FIG. 19B depicts MAPs of the images reconstructed using UBP from signals detected by the two virtual arrays, and the MAPs of the linear test residuals, according to an embodiment. FIG. 19C depicts MAPs of the images reconstructed using the regularized iterative method from signals detected by the two virtual arrays, and the MAPs of the linear test residuals, according to an embodiment.

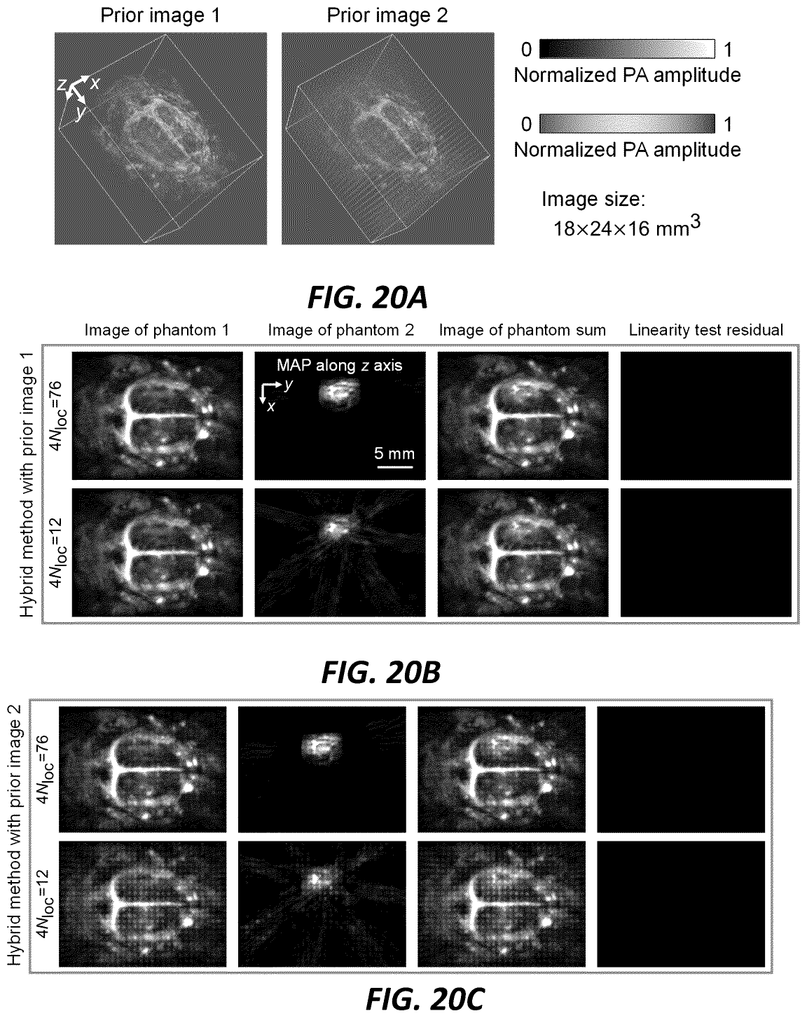

FIGS. 20A-C depict illustrations of the results a linear test of the hybrid method, according to an embodiment, with two prior images and the signals used in FIGS. 19A-C. FIG. 20A depicts a ground-truth image used as the first prior image and a second prior image obtained by adding scattered point sources to the first one, according to an embodiment. FIG. 20B depicts MAPs of the images reconstructed using UBP from signals detected by the two virtual arrays, and the MAPs of the linear test residuals, according to an embodiment. FIG. 20C depicts MAPS of the images reconstructed using the hybrid method with the first and second prior images, respectively, from signals detected by the two virtual arrays (4N_{loc}=76 and 4N_{loc}=12), according to an embodiment.

### G. Numerical Simulations of a Hybrid Method for Fast Functional Imaging

In this Section III (G), numerical simulations are discussed for a hybrid method for fast functional imaging according to an embodiment.

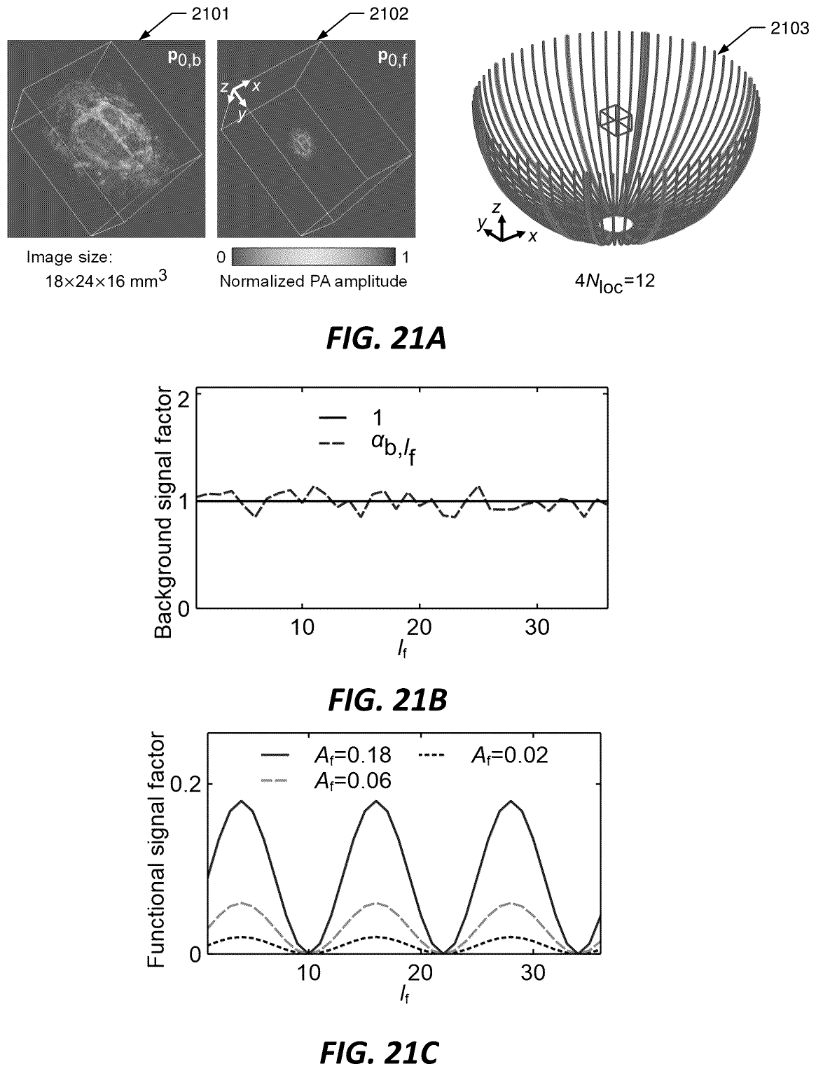

FIGS. 21A-C depict illustrations of numerical phantoms for functional imaging, according to an embodiment. FIG. 21A depicts an image of a background numerical phantom 2101, an image of a functional numerical phantom 2103, and a virtual array 2203 formed by 12 arc arrays, shown as arcs with thick boundaries, respectively, for functional imaging simulation, according to an embodiment. FIG. 21B depicts a plot of modulation factors of the background phantom, according to an embodiment. FIG. 21C depicts a plot of modulation factors of the functional phantom, according to an embodiment.

In one example, numerical phantoms for functional imaging can be obtained using:

$${p}_{0 , {l}_{f}} = {α}_{b , {l}_{f}} {p}_{0 , b} + {α}_{f , {l}_{f}} {p}_{0 , f} , {l}_{f} = 1 , 2 , … , {L}_{f} . \qquad ( Eqn . S36 )$$

Here, p_{0,l}_{_{f}} (e.g., as shown in the image 2102 in FIG. 21A with voxel size of 0.1×0.1×0.1 mm³) is the l_{f}-th numerical phantom; p_{0,b} (e.g., as shown in the image 2101 in FIG. 21A) is the background phantom obtained in imaging with dense sampling and p_{0,f} is the functional phantom obtained from by smoothing, downsampling, and zero padding p_{0,b}; α_{b,l}_{_{f}} and α_{f,l}_{_{f}} are modulation factors of the two phantoms, respectively; and L_{f} is the number of numerical phantoms for functional imaging. The way p_{0,b} is obtained guarantees that the mean value of nonzero voxels in p_{0,b} approximately equals that in p_{0,f}. For simulations in this study, a virtual array formed by 12 arc arrays (e.g., as shown in the image 2103 in FIG. 21A) was used, let L_{f}=36, and let α_{b,l}_{_{f}}˜N (1,0.1), l_{f}=1, 2, . . . , L_{f} (e.g., as shown in FIG. 21B), an amplitude similar to the image relative difference observed in mouse brain functional imaging was used. Also, let:

$${α}_{f , {l}_{f}} = \frac{{A}_{f}}{2} ( sin \frac{6 π ( {l}_{f} - 1 )}{{L}_{f}} + 1 ) , {l}_{f} = 1 , 2 , … , {L}_{f} , \qquad ( Eqn . S37 )$$

Where A_{f} is the functional amplitude. The values of a_{f,l}_{_{f}} with A_{f}=0.18, 0.06, and 0.02 are shown in (e.g., as shown in FIG. 21C and used in the simulations. The forward simulations, image reconstructions (UBP, the regularized iterative method in Eqn. S34, and the hybrid method in Eqn. 4, and functional signal extractions (Eqn. 7) were performed with different λ_{f}. The functional images extracted from four sets of images (ground-truth images and images reconstructed with three methods) using the regularized-correlation-based method with λ_{f}=1.6 are shown in FIG. 22.

FIG. 22 depicts functional images extracted using the regularized-correlation-based method with λ_{f}=1.6 from ground-truth images and images reconstructed with UBP, the regularized iterative method, and the hybrid method for A_{f}=0.18, 0.06, and 0.02, according to an embodiment. The first three rows show both the 3D functional and background images and the last three rows show the MAPs of the functional images along the z-axis. In the first and fourth rows, the true functional regions and examples of false positive regions are indicated by white-solid and white-dotted arrows, respectively. From a comparison of the results, the hybrid method is observed to be superior to both UBP and the regularized iterative method in functional imaging with sparse sampling. The results with other values of λ_{f} also support this observation.

FIG. 23 depicts mouse brain functional images in vivo extracted using the regularized-correlation-based method with λ_{f}=0.02, 0.08, 0.32, 1.28, and 5.12 from images reconstructed with UBP and an example of a hybrid method (4N_{loc}=12), according to an embodiment. The first two rows show both the 3D functional and background images and the last two rows show the MAPs of the functional images along the z-axis. In all images, the true functional regions are indicated by white-solid arrows, and the other high-amplitude functional regions are false positives. Images for λ_{f}=0.32 are highlighted by solid boundaries.

FIG. 24 depicts mouse brain functional images in vivo extracted using the regularized-correlation-based method with λ_{f}=0.02, 0.08, 0.32, 1.28, and 5.12 from images reconstructed through the regularized iterative method (4N_{loc}=40, 20, and 12), according to an embodiment. The first three rows show both the 3D functional and background images and the last three rows show the MAPs of the functional images along the z-axis. In all images, the true functional regions are indicated by white-solid arrows, and the other high-amplitude functional regions are false positives. Images for λ_{f}=0.08 are highlighted by solid boundaries.

### IV. Applications to x-Ray Computed Tomography (CT) and Magnetic Resonance Imaging (MRI)

Although the methods described in Section II are demonstrated in various examples for application with a PACT system, these techniques also apply to X-ray CT and MRI systems according to other implementations. That is, the system matrices in 3D PACT and CT systems correspond to sphere and line integrals, respectively, and the latter can be transformed using Grangeat's method into plane integrals, which are locally equivalent to sphere integrals. System matrices in 2D PACT and CT correspond to circle (reduced from a sphere) and line integrals, respectively, which are locally equivalent. MRI is more complex due to its high flexibility ink-space sampling. For radial sampling MRI, the acquired signals can be transformed to integrals on lines and planes, respectively, for 2D and 3D imaging using the Fourier slice theorem. For other sampling patterns, further analysis may disclose proper transformations to obtain integrals that are locally equivalent to those in PACT. The methods described herein may rely on efficient slicing and manipulation of the system matrix, which is directly available for CT where the system matrix is highly sparse and is achieved in PACT through system matrix compression based on SVD and FFT as discussed in Section II (A). For radial sampling MRI, the k-space signals can be transformed to line or plane integrals and compressed through SVD and FFT. For general MRI, further study may transform the k-space signals to compressible integrals. In summary, through certain transformations, system matrices in CT, MRI, and PACT share similar local structures, which allows the methods discussed in Section II to be transferred to CT and MRI.

### V. Examples of Systems

The TISMC and hybrid methods described above may be implemented using one or more computing devices (e.g., computing device 2520 in FIG. 25A or computing device 2522 in FIG. 25B). For example, a method may be performed using a computational device such as a server device, a laptop computer, a desktop computer, or the like.

FIG. 25A depicts a block diagram of an example of components of tomographic imaging system 2500 for performing TISMC methods and/or hybrid methods, according to various embodiments. Tomographic imaging system 2500 includes a tomographic imaging device 2510 configured to acquire tomographic images. Some examples of suitable tomographic imaging devices include a PACT system, a CT system, an MRI system, and ultrasound computed tomography (USCT) system. Tomographic imaging device 2510 includes one or more sensor arrays 2512 (e.g., ultrasonic transducer arrays in PACT and USCT, X-ray detection arrays in CT, and RF-coils in MRI). Tomographic imaging system 2500 also includes a computing device 2520 in electrical communication with tomographic imaging device 2510 to receive signals detected by the one or more sensor arrays 2512 such as, e.g., sparsely sampled signals and/or densely sampled signals. In some cases, the computing device 2520 may also send control signals to the tomographic imaging device 2510 to control functions (e.g., sampling) of the tomographic imaging device 2510. Communication between components of tomographic imaging system 2500 may be in wireless and/or wired form. The computing device 2520 may include one or more processors (e.g., a CPU, GPU or computer, analog and/or digital input/output connections, controller boards, etc.) and a non-transitory computer readable medium in electrical communication with the one or more processors.

FIG. 25B depicts components of a computing device 2522, according to embodiments. In one implementation, the computing device 2520 in FIG. 25A includes one or more of the components of the computing device 2522 in FIG. 25B. In various examples, computing device 2522 is in electrical communication with a tomographic imaging device (e.g., tomographic imaging device 2510 in FIG. 25A) to receive signals with tomographic data such as sparsely sampled signals and/or densely sampled signals. In some cases, the computing device 2522 may also send control signals to the tomographic imaging device to control functions of the tomographic imaging device. Communication between components of computing device 2522 may be in wireless and/or wired form.

In FIG. 25B, computing device 2522 includes a bus 2523 coupled to an input/output (I/O) subsystem 2532, one or more processors 2534, one or more communication interfaces 2536, a main memory 2538, a secondary memory 2542, and a power supply 2540. One of more of these components may be in separate housing.

I/O subsystem 2532, includes, or is in communication with, one or more components, which may implement an interface for interacting with human users and/or other computer devices depending upon the application. Certain embodiments disclosed herein may be implemented in program code on computing device 2522 with I/O subsystem 2532 used to receive input program statements and/or data from a human user (e.g., via a graphical user interface (GUI), a keyboard, touchpad, etc.) and to display them back to the user, for example, on a display. The I/O subsystem 2532 may include, e.g., a keyboard, mouse, graphical user interface, touchscreen, or other interfaces for input, and, e.g., an LED or other flat screen display, or other interfaces for output. Other elements of embodiments may be implemented with a computer device like that of computing device 2522 without I/O subsystem 2532. According to various embodiments, the one or more processors 2534 may include a CPU, GPU or computer, analog and/or digital input/output connections, controller boards, etc.

Program code may be stored in non-transitory computer readable media such as secondary memory 2542 or main memory 2538 or both. The one or more processors 2534 may read program code from one or more non-transitory media and execute the code to enable computing device 2522 to accomplish the methods performed by various embodiments described herein, such as TISMC and/or hybrid method. Those skilled in the art will understand that the one or more processors 2534 may accept source code and interpret or compile the source code into machine code that is understandable at the hardware gate level of the one or more processors 2534.

Communication interfaces 2536 may include any suitable components or circuitry used for communication using any suitable communication network (e.g., the Internet, an intranet, a wide-area network (WAN), a local-area network (LAN), a wireless network, a virtual private network (VPN), and/or any other suitable type of communication network). For example, communication interfaces 2536 can include network interface card circuitry, wireless communication circuitry, etc.

In certain embodiments, computing device 2522 may be part of or connected to a controller that is employed to control functions of a tomographic imaging device (e.g., tomographic imaging device 2510 such as controlling data acquisition by one or more sensors (e.g., one or more sensor arrays 2512 in FIG. 25A). The controller will typically include one or more memory devices and one or more processors. The processor may include a CPU or computer, analog and/or digital input/output connections, stepper motor controller boards, etc.

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C #, C++ or Python, Matlab, or other suitable language/computational software, including low level code, including code written for field programmable gate arrays, for example in VHDL; embedded artificial intelligence computing platform, for example in Jetson. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random-access memory (RAM), a read only memory (ROM), a magnetic media such as a hard-drive or a floppy disk, or an optical media such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

1. A tomographic imaging system matrix compression method, comprising:
(a) acquiring a plurality of detected tomographic system responses;
(b) shifting one or more of the detected tomographic system responses to align arrival times;
(c) for each virtual sensor of a plurality of virtual sensors, determining an approximate system response that is a linear combination of a plurality of basis functions, wherein the approximate system response is determined by performing a singular value decomposition (SVD) process on the aligned, detected tomographic system responses to obtain a plurality of coefficients for the linear combination; and
(d) determining a compressed system matrix including approximate system responses for all virtual sensors from the plurality of coefficients determined for each of the virtual sensors.

2. The tomographic imaging system matrix compression method of claim 1, wherein the plurality of basis functions comprises a set of three temporal singular functions obtained in the SVD process on the aligned, detected tomographic system responses.

3. The tomographic imaging system matrix compression method of claim 1, wherein (b) comprises shifting one or more of the detected tomographic system responses to align to arrival times of the detected tomographic system responses and the basis functions.

4. (canceled)

5. The tomographic imaging system matrix compression method of claim 1, wherein each sensor of the plurality of virtual sensors is one of an ultrasonic transducer, an X-ray radiation sensor, or a magnetic resonance sensor.

6. The tomographic imaging system matrix compression method of claim 1, wherein the plurality of virtual sensors comprises a plurality of physical sensors rotated/translated to a plurality of locations of the virtual sensors during operation.

7-8. (canceled)

9. The tomographic imaging system matrix compression method of claim 1, further comprising obtaining detected signals detected by a plurality of sensors of a tomographic imaging system.

10. (canceled)

11. The tomographic imaging system matrix compression method of claim 9, further comprising: (i) applying the plurality of coefficients from the compressed system matrix to the detected signals to generate a time series of coefficients for a simulated signal for each virtual sensor of the plurality of virtual sensors.

12. The tomographic imaging system matrix compression method of claim 11, further comprising repeating (i) for each voxel-virtual sensor combination.

13. The tomographic imaging system matrix compression method of claim 11, further comprising generating a simulated signal for each virtual sensor from a linear combination of the plurality of basis functions using the time series of coefficients.

14. The tomographic imaging system matrix compression method of claim 13, further comprising performing an iterative reconstruction procedure using the simulated signals from all the virtual sensors to reconstruct a tomographic image.

15-16. (canceled)

17. A system comprising:
a tomographic imaging device comprising one or more sensor arrays;
one or more processors in electrical communication with the tomographic imaging device; and
one or more processor-readable media storing instructions which, when executed by the one or more processors, cause performance of:
(a) performing piece-wise compression of a plurality of aligned system responses to generate a compressed system matrix;
(b) applying the compressed system matrix to generate a plurality of simulated virtual sensor signals; and
(c) performing an iterative reconstruction procedure to reconstruct tomographic image from detected sensor signals by comparing a difference between the simulated virtual sensor signals and the detected sensor signals.

18. The system of claim 17, wherein (a) comprises:
(i) acquiring a plurality of detected tomographic system responses;
(ii) shifting one or more of the detected tomographic system responses to align arrival times to obtain the plurality of aligned system responses;
(iii) for each virtual sensor element of a plurality of virtual sensor elements, determining an approximate system response that is a linear combination of a plurality of basis functions, wherein the approximate system response is determined by performing a singular value decomposition (SVD) process on the aligned system responses to obtain a plurality of coefficients for the linear combination; and
(iv) determining a compressed system matrix including approximate system responses for all virtual sensors from the plurality of coefficients determined for each of the virtual sensors.

19. The system of claim 18, wherein the plurality of basis functions comprises a first three temporal singular functions.

20. The system of claim 18, wherein the plurality of virtual sensor elements corresponds to a plurality of detection locations of sensor elements of the one or more sensor arrays during rotation/translation during operation.

21. The system of claim 17, wherein the one or more sensor arrays comprises at least one of an ultrasonic transducer, an X-ray radiation sensor, or a magnetic resonance sensor.

22. (canceled)

23. The system of claim 17, wherein the one or more sensor arrays comprises a plurality of ultrasonic arc transducer arrays.

24. The system of claim 17, wherein the tomographic imaging device is one of a photoacoustic computed tomography system, an X-ray computed tomography system, or a magnetic resonance imaging system.

25. (canceled)

26. A method for functional tomographic imaging, the method comprising:
(a) performing iterative reconstruction of a smooth modulation image using a set of sparsely sampled functional signals from a plurality of sensors of a tomographic imaging device;
(b) obtaining a modulated prior image by upsampling the smooth modulation image and multiplying the upsampled smooth modulation image by a densely sampled prior image;
(c) applying a dense-sampling system matrix to the modulated prior image to determine a dense forward solution and reconstructing a modulated universal back-projection (UBP) image from the dense forward solution using a UBP method;
(d) applying a sparse sampling system matrix to the modulated prior image to determine a sparsely sampled solution and reconstructing a residual image using the UBP method from a difference between the set of sparsely sampled functional signals and the sparsely sampled solution; and
(e) determining a hybrid functional image by adding the modulated UBP image to the residual image.

27. The method for functional tomographic imaging of claim 26, further comprising
prior to (a), acquiring the set of densely sampled signals and reconstructing the densely sampled prior image from the set of densely sampled signals; and
prior to (a), acquiring a plurality of sets of sparsely sampled functional signals from the plurality of sensors of the tomographic imaging system.

28-32. (canceled)

33. A system comprising:
a tomographic imaging device comprising one or more sensor arrays;
one or more processors in electrical communication with the tomographic imaging device; and
one or more processor-readable media storing instructions which, when executed by the one or more processors, cause performance of:
(a) performing iterative reconstruction of a smooth modulation image using a set of sparsely sampled functional signals from a plurality of sensors of a tomographic imaging device;
(b) obtaining a modulated prior image by upsampling the smooth modulation image and multiplying the upsampled smooth modulation image by a densely sampled prior image;
(c) applying a dense-sampling system matrix to the modulated prior image to determine a dense forward solution and reconstructing a modulated universal back-projection (UBP) image from the dense forward solution using a UBP method;
(d) applying a sparse sampling system matrix to the modulated prior image to determine a sparsely sampled solution and reconstructing a residual image using the UBP method from a difference between the set of sparsely sampled functional signals and the sparsely sampled solution; and
(e) determining a hybrid functional image by adding the modulated UBP image to the residual image.

34. The system of claim 33, wherein the one or more sensor arrays comprises at least one of an ultrasonic transducer, an X-ray radiation sensor, or a magnetic resonance sensor.

35. (canceled)
